(19) United States
(12) Patent Application Publication
(10) Pub. No.: US 2017/0247758 A1
(43) Pub. Date: Aug. 31, 2017

(54) METHODS, COMPUTER-READABLE MEDIA, AND SYSTEMS FOR ASSESSING SAMPLES AND WOUNDS, PREDICTING WHETHER A WOUND WILL HEAL, AND MONITORING EFFECTIVENESS OF A TREATMENT

(71) Applicant: DREXEL UNIVERSITY, Philadelphia, PA (US)

(72) Inventors: Kara SPILLER, Glenside, PA (US); Sina NASSIRI, Philadelphia, PA (US); Michael WEINGARTEN, Penn Valley, PA (US)

(73) Assignee: DREXEL UNIVERSITY, Philadelphia, PA (US)

(21) Appl. No.: 15/500,910

(22) PCT Filed: Aug. 14, 2015
(86) PCT No.: PCT/US15/45303

§ 371 Date: Jan. 31, 2017

Related U.S. Application Data
(60) Provisional application No. 62/038,584, filed on Aug. 18, 2014.
(60) Provisional application No. 62/104,032, filed on Jan. 15, 2015.
(60) Provisional application No. 62/179,175, filed on Apr. 29, 2015.

(51) Int. Cl. C12Q 1/68; A61K 38/21; G01N 33/569

(52) U.S. Cl. CPC C12Q 1/6881; G01N 33/56972; C12Q 1/6886; A61K 38/217; C12Q 2600/118; C12Q 2600/158; G01N 2800/52

(57) ABSTRACT

One aspect of the invention provides a method of predicting whether a wound will heal. The method includes: obtaining a first measurement of a first macrophage phenotype population within a first sample obtained from a wound; obtaining a second measurement of a second macrophage phenotype population from the wound, wherein the second measurement of the second macrophage phenotype population is either: a different macrophage phenotype obtained from the first sample or the same macrophage phenotype obtained from a second, later sample from the wound; comparing the first measurement to the second measurement; and predicting whether the wound will heal based on a result of the comparing step.

### CROSS-REFERENCE TO RELATED APPLICATION

This application claims priority to U.S. Provisional Patent Application Ser. No. 62/038,584, filed Aug. 18, 2014, U.S. Provisional Patent Application Ser. No. 62/104,032, filed Jan. 15, 2015, and U.S. Provisional Patent Application Ser. No. 62/179,175, filed Apr. 29, 2015. The entire content of each of these applications is hereby incorporated herein by reference in its entirety.

### BACKGROUND OF THE INVENTION

Dysfunctional wound healing is a major complication of both type 1 and type 2 diabetes. Foot ulcerations, which occur in 15% of diabetic patients, lead to over 82,000 lower limb amputations annually in the United States, with a direct cost of $5 billion per year. The selection of an appropriate treatment strategy from dozens of choices available on the market, and knowing when to discontinue an ineffective treatment in favor of a different one, is critical to success. However, the process of wound healing is complex and difficult to assess. Currently, the gold standard of distinguishing between healing and nonhealing is based on physician observation and wound size measurement. These methods are very subjective and prone to error, with only 58% positive predictive value.

### SUMMARY OF THE INVENTION

One aspect of the invention provides a method of predicting whether a wound will heal. The method includes: obtaining a first measurement of a first macrophage phenotype population within a first sample obtained from a wound; obtaining a second measurement of a second macrophage phenotype population from the wound, wherein the second measurement of the second macrophage phenotype population is either: a different macrophage phenotype obtained from the first sample or the same macrophage phenotype obtained from a second, later sample from the wound; comparing the first measurement to the second measurement; and predicting whether the wound will heal based on a result of the comparing step.

This aspect of the invention can have a variety of embodiments. The first measurement and the second measurement can be derived from gene expression data.

The first macrophage phenotype and the second macrophage phenotype can be M1. The first measurement and the second measurement can be gene expression values for a single marker associated with M1 macrophage activity. The single marker associated with M1 macrophage activity can be selected from the group consisting of: CCR7, CD80, IL1B, and VEGF.

The first macrophage phenotype and the second macrophage phenotype can be M2. The first measurement and the second measurement can be gene expression values for a single marker associated with M2 macrophage activity. The single marker associated with M2 macrophage activity can be selected from the group consisting of: CCL18, CD163, CD206, MDC, PDGF, and TIMP3.

The first macrophage phenotype and the second macrophage phenotype is M2c. The first measurement and the second measurement can be gene expression values for a single marker associated with M2c macrophage activity. The single marker associated with M2c macrophage activity can be selected from the group consisting of: CD163, MMP7, TIMP1, VCAN, PLAU, PROS1, MMP8, SRPX2, NAIP, F5, SEMA6B, SH3PXD2B, SLC25A19, COL22A1, SLC12A8, FPR1, PDPN, LIN7A, GLDN, CD226, PTPRN, TSPAN13, PCOLCE2, LIMCH1, PLOD2, CD300E, CASC15, LGI2, SH2D4A, CXADR, GXYLT2, WASF1, NPDC1, DNAH17, SPINK1, PARVA, CLEC1A, TDO2, LAMC2, CCR2, GRPR, CD163L1, FGD1, EDNRB, KIAA1211L, PCDGA11, PHEX, CRYAB, AR, PVALB, NMNAT2, SL16A2, FAP, C10orf55, BNIP3P1, DDAH1, BICC1, SPATA20P1, C7orf63, CHRNA6, BCYRN1, ZFPM2, PRL, CHGA, LRRC2, DNAH17-AS1, OR13A1, PRG3, RNF175, PROK2, AWAT2, SNCB, and KCNK15.

The first macrophage phenotype can be selected from the group consisting of M1, M2, M2a, M2b, and M2c; and the second macrophage phenotype can be selected from the group consisting of M1, M2, M2a, M2b, and M2c.

The first measurement and the second measurement can be functions of gene expression values for a plurality of markers. The functions can be weighted summations. The weighted summations can utilize weighting coefficients obtained from principal component analysis. The weighted summations can utilize weighting coefficients obtained through optimization. The weighted summations can utilize weighting coefficients obtained through machine learning techniques. The weighted summations can utilize one or more selected from the group consisting of: a t statistic obtained from a Student's t-test for corresponding markers between M1 and M2 macrophages cultured in vitro as weighting coefficients, weighting coefficients that minimize a p value of a t-test performed on a weighted summation of M1 and M2 macrophages cultured in vitro, weighting coefficients obtained from using a mean-centering method, and weighting coefficients that are equal to each other. The functions can be non-linear functions.

The sample can be obtained from the wound via debriding.

Another aspect of the invention provides a method of assessing a sample. The method includes: calculating a first ratio of M1 macrophages to M2 macrophages in a first sample based on gene expression values for at least one marker associated with M1 macrophage activity and at least one marker associated with M2 macrophage activity.

This aspect of the invention can have a variety of embodiments. The first ratio can be a ratio of a first gene expression value for a single marker associated with M1 macrophage activity to a second gene expression value for a single marker associated with M2 macrophage activity. The single marker associated with M1 macrophage activity can be selected from the group consisting of: CCR7, CD80, IL1B, and VEGF. The single marker associated with M2 macrophage activity can be selected from the group consisting of: CCL18, CD163, CD206, MDC, PDGF, and TIMP3. The single marker associated with M1 macrophage activity can be IL1B and wherein the single marker associated with M2 macrophage activity can be CD206. The single marker associated with M1 macrophage activity can be IL1B and wherein the single marker associated with M2 macrophage activity can be CD163.

The M2 macrophages can be M2c macrophages and the at least one marker associated with M2 macrophage activity can be selected from the group consisting of: CD163, MMP7, TIMP1, VCAN, PLAU, PROS1, MMP8, SRPX2, NAIP, F5, SEMA6B, SH3PXD2B, SLC25A19, COL22A1, SLC12A8, FPR1, PDPN, LIN7A, GLDN, CD226, PTPRN, TSPAN13, PCOLCE2, LIMCH1, PLOD2, CD300E, CASC15, LGI2, SH2D4A, CXADR, GXYLT2, WASF1, NPDC1, DNAH17, SPINK1, PARVA, CLEC1A, TDO2, LAMC2, CCR2, GRPR, CD163L1, FGD1, EDNRB, KIAA1211L, PCDGA11, PHEX, CRYAB, AR, PVALB, NMNAT2, SL16A2, FAP, C10orf55, BNIP3P1, DDAH1, BICC1, SPATA20P1, C7orf63, CHRNA6, BCYRN1, ZFPM2, PRL, CHGA, LRRC2, DNAH17-AS1, OR13A1, PRG3, RNF175, PROK2, AWAT2, SNCB, and KCNK15.

The calculating step can include: calculating a first function of gene expression values of each of a first plurality of markers associated with M1 macrophages; and calculating a second function of gene expression values of each of a second plurality of markers associated with M2 macrophages. The first function can be a first weighted summation and the second function can be a second weighted summation. The first weighted summation and the second weighted summation can utilize weighting coefficients obtained from principal component analysis. The first weighted summation and the second weighted summation can utilize weighting coefficients obtained through optimization. The first weighted summation and the second weighted summation can utilize weighting coefficients obtained through machine learning techniques. The first weighted summation and the second weighted summation can utilize a t statistic obtained from a Student's t-test for corresponding markers between M1 and M2 macrophages cultured in vitro as weighting coefficients. The first weighted summation and the second weighted summation can utilize weighting coefficients that minimize a p value of a t-test performed on a weighted summation of M1 and M2 macrophages cultured in vitro. The first weighted summation and the second weighted summation can utilize weighting coefficients obtained from using a mean-centering method. The first weighted summation and the second weighted summation can utilize weighting coefficients that are equal to each other. The first function and the second function can be non-linear functions.

The method can further include: calculating a second ratio of M1 macrophages to M2 macrophages in a second sample based on gene expression values for at least one marker associated with M1 macrophage activity and at least one marker associated with M2 macrophage activity, the second sample obtained from a same source as the first sample after passage of a period of time; and comparing the second ratio to the first ratio. The comparing step can include calculating a fold change from the first ratio to the second ratio. The comparing step can include one or more selected from the group consisting of: an absolute difference and a rate of change. The period of time can be selected from the group consisting of: at least 1 day, at least 2 days, at least 3 days, at least 4 days, at least 5 days, at least 6 days, at least 7 days, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, and at least 16 weeks. The method can further include correlating an increase or substantial similarity between the first ratio and the second ratio with a nonhealing condition. The method can further include correlating a decrease from the first ratio to the second ratio with a healing condition.

The sample can be a biological sample. The sample can be obtained from a wound. The sample can be obtained during an initial medical encounter concerning the wound. The sample can be obtained from a location adjacent to an implanted medical device. The sample can be obtained from a blood vessel. The sample can be selected from the group consisting of: an artery, a vein, and a capillary.

The method can further include: calculating a second ratio of M1 macrophages to M2 macrophages in a second sample based on gene expression values for at least one marker associated with M1 macrophage activity and at least one marker associated with M2 macrophage activity, the second sample obtained from a different source than the first sample, wherein the first sample and the second sample are obtained adjacent to first and second materials, respectively, in a testing environment; and comparing the second ratio to the first ratio. The testing environment can be selected from the group consisting of: an in vitro testing environment and an in vivo testing environment.

Another aspect of the invention provides a non-transitory computer readable medium containing computer-readable program code including instructions for performing the methods described herein.

Another aspect of the invention provides a system including: a gene expression device; and a processor programmed to implement the methods described herein.

This aspect of the invention can have a variety of embodiments. The gene expression device can be selected from the group consisting of: a thermocycler, a microarray, and an RNA Sequencing (RNA-seq) device.

Another aspect of the invention provides a method of assessing a wound. The method includes: extracting RNA from debrided wound tissue; measuring expression of one or more genes within the RNA; and calculating a ratio of M1 macrophages to M2 macrophages based on the measured gene expression.

This aspect of the invention can have a variety of embodiments. The debrided wound tissue can be removed from a dressing previously applied a wound. The debrided wound tissue can be from one or more selected from the group consisting of: a diabetic ulcer, a pressure ulcer, a chronic venous ulcer, a burn, a wound caused by an autoimmune disease, a wound caused by Crohn's disease, a wound caused by atherosclerosis, a tumor, a medical implant insertion point, a surgical wound, a bone fracture, a tissue tear, and a tissue rupture. The measuring expression step can include using one or more tools or techniques selected from the group consisting of: cDNA synthesis, quantitative PCR (qPCR), microarrays, and RNA Sequencing (RNA-seq).

Another aspect of the invention provides a high-throughput screening system including: a measurement device; and a data processor programmed to implement the method described herein.

Another aspect of the invention provides a method of monitoring effectiveness of a treatment of a non-healing wound. The method includes: administering to a patient a therapeutic agent designed to treat a non-healing wound; obtaining a first measurement of a first macrophage phenotype population within a first sample obtained from the non-healing wound; obtaining a second measurement of second macrophage phenotype population from the non-healing wound, wherein the second measurement of the second macrophage phenotype population is either: a different macrophage phenotype obtained from the first sample; or the same macrophage phenotype obtained from a second, later sample from the non-healing wound; comparing the first measurement to the second measurement; and assessing whether the treatment of the non-healing wound is effective based on a result of comparing the measurements.

This aspect of the invention can have a variety of embodiments. The therapeutic agent can be selected from the group consisting of an L-arginine, hyperbaric oxygen, a moist saline dressing, an isotonic sodium chloride gel, a hydroactive paste, a polyvinyl film dressing, a hydrocolloid dressing, a calcium alginate dressing, and a hydrofiber dressing. The treatment can be a low-intensity ultrasound treatment.

The method can further include comparing an M1/M2 ratio with a threshold value that discriminates between wound healing and non-wound healing and adjusting the treatment based on the M1/M2 ratio, wherein: if the M1/M2 ratio is at or below the threshold value, the administration of therapeutic agent is increased, and if the M1/M2 ratio is above the threshold value, the administration of the therapeutic agent is not increased. If the level is at or below the threshold value, the therapeutic agent can be replaced by a different therapeutic agent.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1A depicts a method of assessing a sample according to an embodiment of the invention.

FIG. 1B depicts transcriptional profiling of macrophages polarized in vitro to the M1 or M2 phenotypes.

FIG. 1C depicts a linearly-summed M1 over M2 score applied to in vitro polarized macrophages (mean+/−SEM, n=5-6). Statistical significance was determined using unpaired two-sided Student's t test (*P<0.05).

FIG. 1D depicts the change in M1 over M2 score (relative to normal skin) over time, in healing acute wounds (mean+/−SEM, n=3 pooled data from 15 samples), using data obtained from J. A. Greco et al., “A microarray analysis of temporal gene expression profiles in thermally injured human skin,” 36(2) Burns 192-204 (March 2010) (hereinafter “Greco”).

FIG. 1E depicts the change in M1 over M2 score (relative to first time point) over time, in healing vs. nonhealing diabetic wounds over 4 weeks from the initial visit (mean+/−SEM, n=3-4).

FIG. 1F depicts a comparison of mean fold change of M1 over M2 score (relative to first time point), between healing and nonhealing diabetic ulcers at 4 weeks (mean+/−SEM, n=3-4). Statistical significance was analyzed using unpaired two-sided Student's t test (**P<0.01).

FIG. 1G depicts raw gene expression data over time for a typical healing wound.

FIG. 1H depicts raw gene expression data over time for a typical nonhealing wound.

FIG. 2 depicts changes in wound size over 30 days, expressed as fold change over day zero. Panels (a)-(d) depict the nonhealing group. Panels (e)-(g) the healing group. Panel (h) depicts the comparison between nonhealing and healing groups at 4 weeks.

FIG. 3 depicts box and whisker plot (using the Tukey method) of gene expression data for individual markers of M1 and M2 macrophages cultivated in vitro.

FIG. 4 depicts principal component analysis of gene expression data of macrophages cultivated in vitro. Panel (a) depicts a PCA biplot. Panel (b) depicts a PCA sample plot, which is a scatterplot of transformed data using first and second principal components.

FIG. 5 depicts the effects of applying PCA weighting to the gene expression data. Panel (a) depicts the effect of applying PCA weighting to gene expression data of macrophages cultivated in vitro. Panel (b) depicts the effect of applying PCA weighting to gene expression data from chronic diabetic wounds at 4 weeks. Panel (c) depicts the effect of applying PCA weighting to gene expression data from healing acute wounds. Panels (d)-(g) depict the M1/M2 score as calculated using PCA weighting over time for the nonhealing group. Panels (h)-(j) depict the M1/M2 score as calculated using PCA weighting over time for the healing group.

FIG. 6 depicts the effects of applying weighted scaling to the gene expression data. Panel (a) depicts the effect of applying weighted scaling to gene expression data of macrophages cultivated in vitro. Panel (b) depicts the effect of applying weighted scaling to gene expression data from chronic diabetic wounds at 4 weeks. Panel (c) depicts the effect of applying weighted scaling to gene expression data from healing acute wounds. Panels (d)-(g) depict the M1/M2 score as calculated using weighted scaling weighting over time for the nonhealing group. Panels (h)-(j) depict the M1/M2 score as calculated using weighted scaling over time for the healing group.

FIG. 7 depicts the effects of applying a greedy method to weight the gene expression data. Panel (a) depicts the effect of applying a greedy method to weight the gene expression data of macrophages cultivated in vitro. Panel (b) depicts the effect of applying a greedy method to weight the gene expression data from chronic diabetic wounds at 4 weeks. Panel (c) depicts the effect of applying a greedy method to weight the gene expression data from healing acute wounds. Panels (d)-(g) depict the M1/M2 score as calculated using a greedy method to weight the gene expression data over time for the nonhealing group. Panels (h)-(j) depict the M1/M2 score as calculated using a greedy method to weight the gene expression data over time for the healing group.

FIG. 8 depicts the effects of applying a mean-centering method to weight the gene expression data. Panel (a) depicts the effect of applying a mean-centering method to weight the gene expression data of macrophages cultivated in vitro. Panel (b) depicts the effect of applying a mean-centering method to weight the gene expression data from chronic diabetic wounds at 4 weeks. Panel (c) depicts the effect of applying a mean-centering method to weight the gene expression data from healing acute wounds. Panels (d)-(g) depict the M1/M2 score as calculated using a mean-centering method to weight the gene expression data over time for the nonhealing group. Panels (h)-(j) depict the M1/M2 score as calculated using a mean-centering method to weight the gene expression data over time for the healing group.

FIG. 9 depicts the effects of applying a linear sum method to weight the gene expression data. Panel (a) depicts the effect of applying a linear sum method to weight the gene expression data of macrophages cultivated in vitro. Panel (b) depicts the effect of applying a linear sum method to weight the gene expression data from chronic diabetic wounds at 4 weeks. Panel (c) depicts the effect of applying a linear sum method to weight the gene expression data from healing acute wounds. Panels (d)-(g) depict the M1/M2 score as calculated using a linear sum method to weight the gene expression data over time for the nonhealing group. Panels (h)-(j) depict the M1/M2 score as calculated using a linear sum method to weight the gene expression data over time for the healing group.

FIG. 10A depicts the effects of considering only IL1B gene expression over CD206 gene expression. Panel (a) depicts the effect of considering only IL1B over CD206 gene expression data for macrophages cultivated in vitro. Panel (b) depicts the effect of considering only IL1B over CD206 gene expression data from chronic diabetic wounds at 4 weeks. Panel (c) depicts the effect of considering only IL1B over CD206 gene expression data from healing acute wounds. Panels (d)-(g) depict the M1/M2 score as calculated considering only IL1B over CD206 gene expression data over time for the nonhealing group. Panels (h)-(j) depict the M1/M2 score as calculated considering only IL1B over CD206 gene expression data over time for the healing group.

FIG. 10B depicts the effects of considering only IL1B gene expression over CD163 gene expression. Panel (a) depicts the effect of considering only IL1B over CD163 gene expression data for macrophages cultivated in vitro. Panel (b) depicts the effect of considering only IL1B over CD163 gene expression data from chronic diabetic wounds at 4 weeks. Panel (c) depicts the effect of considering only IL1B over CD163 gene expression data from healing acute wounds. Panels (d)-(g) depict the M1/M2 score as calculated considering only IL1B over CD163 gene expression data over time for the nonhealing group. Panels (h)-(j) depict the M1/M2 score as calculated considering only IL1B over CD163 gene expression data over time for the healing group.

FIG. 11 provides assessment and comparison of methods in prediction of healing outcomes. Panel (a) depicts a profile analysis of fold change of wound size over day 0, comparing nonhealing and healing chronic diabetic wounds. Panel (b) depicts a profile analysis of fold change of the M1/M2 score calculated using a PCA method to weight the gene expression data over day 0, comparing nonhealing and healing chronic diabetic wounds. Panel (c) provides a graphical representation of the true positive rate (TPR) vs. the false positive rate (FPR) over the course of 4 weeks, using the PCA method as a diagnostic assay. Panel (d) depicts a profile analysis of fold change of the M1/M2 score calculated using a weighted scaling method to weight the gene expression data over day 0, comparing nonhealing and healing chronic diabetic wounds. Panel (e) provides a graphical representation of TPR vs. FPR over the course of 4 weeks, using the weighted scaling method as a diagnostic assay. Panel (f) depicts a profile analysis of fold change of M1/M2 score calculated using a greedy method to weight the gene expression data over day 0, comparing nonhealing and healing chronic diabetic wounds. Panel (g) provides a graphical representation of TPR vs. FPR over the course of 4 weeks, using greedy method as a diagnostic assay. Panel (h) depicts a profile analysis of fold change of the M1/M2 score calculated using a mean-centering method to weight the gene expression data over day 0, comparing nonhealing and healing chronic diabetic wounds. Panel (i) provides a graphical representation of TPR vs. FPR over the course of 4 weeks, using a mean-centering method as a diagnostic assay. Panel (j) depicts a profile analysis of fold change of the M1/M2 score calculated using a linear sum method to weight the gene expression data over day 0, comparing nonhealing and healing chronic diabetic wounds. Panel (k) provides a graphical representation of TPR vs. FPR over the course of 4 weeks, using a linear sum method as a diagnostic assay. Panel (1) depicts a profile analysis of fold change of M1/M2 score calculated using only IL1B over CD206 gene expression data over day 0, comparing nonhealing and healing chronic diabetic wounds. Panel (m) provides a graphical representation of TPR vs. FPR over the course of 4 weeks, using an IL1B/CD206 method as a diagnostic assay.

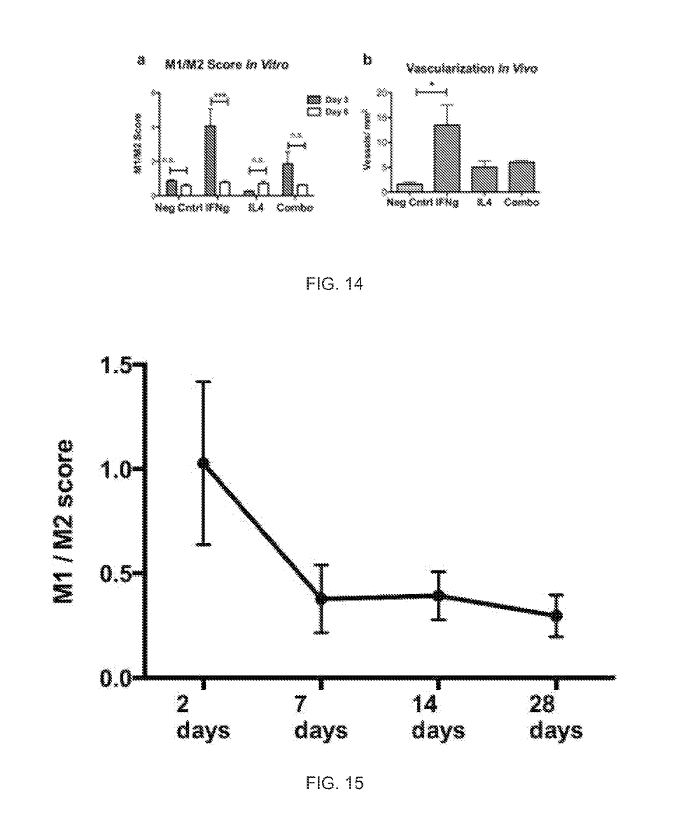

FIG. 12 provides a correlation plot of the gene expression data of macrophages cultured in vitro. Similar to a correlation matrix, a correlation plot is diagonally symmetric. Positive and negative correlations are depicted by the slope of the major axes of the corresponding ellipses. The higher the correlation factor, the closer the corresponding ellipse to a perfect line.

FIG. 13 depicts a system 1300 for assessing a wound according to an embodiment of the invention.

FIG. 14 depicts bar graphs of M1/M2 scores in vitro and vascularization in vivo for different biomaterials according to an embodiment of the invention.

FIG. 15 depicts M1/M2 scores over time after stent implantation according to an embodiment of the invention.

FIG. 16 depicts raw gene expression data over time after stent implantation.

FIG. 17 depicts the M1 over M2 score in healing and nonhealing diabetic ulcers over time.

FIG. 18 depicts a method of predicting whether a wound will heal according to an embodiment of the invention.

FIGS. 19A, 19B, and 19C depict volcano plots showing genes that are up- and down-regulated in M2c macrophages relative to M0 macrophages, M1 macrophages, and M2a macrophages, respectively. FIGS. 19D and 19E depict Venn diagrams of overlapping and distinct genes that are up-regulated and down-regulated, respectively, in M1, M2a, and M2c macrophages relative to M0 macrophages.

FIGS. 20A and 20B depicts transcriptional profiles across M0, M1, M2a, and M2c macrophages for biomarkers of M1, M2a, and M2c macrophages.

FIG. 21 depicts bar graphs of protein secretion (as determined by ELISA analysis of cell culture supernatant) for newly discovered M2c markers TIMP1, MMP7, and MMP8.

FIG. 22 depicts bar graphs of summed expression of raw data of ˜5 highly expressed genes of the M1, M2a, and M2c phenotypes in publicly available data.

FIG. 23 depicts heat maps showing that M1 markers are upregulated in the early phases of wound healing while M2c markers are upregulated at later stages of wound healing in publicly available data.

FIG. 24 depicts bar graphs showing that the M1 marker SOD2 is upregulated at early times after injury while the M2c marker CD163 is increasingly upregulated at over time after injury.

FIG. 25 depicts a method 2500 of predicting tumor progression according to an embodiment of the invention.

FIG. 26 depicts a transient increase in M1 over M2 score (relative to a first time point) in wounds treated with low-intensity ultrasound vs. nontreated diabetic wounds over 4 weeks from the initial visit.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the terms “comprises,” “comprising,” “containing,” “having,” and the like can have the meaning ascribed to them in U.S. patent law and can mean “includes,” “including,” and the like.

As used herein, the term “healing” refers to the process by which a body repairs itself after injury. The healing process can include several stages such as hemostasis (blood clotting), inflammation, proliferation (growth of new tissue), and maturation (remodeling). Embodiments of the invention can be used to make predictions regarding whether the wound will progress through all or the rest of the healing process without the need for enhanced techniques or can be utilized to make predictions regarding whether wound will progress to a particular stage of healing (e.g., proliferation) without the need for enhanced techniques.

As used herein, the term “high-throughput screening” refers to a screening method or system that allows analysis of a large number of samples by analyzing the presence, absence, relative levels, or response in one or more measurements including, but not limited to, nucleic acid makeup, gene expression, protein levels, functional activity, response to a stimulus, etc.

The terms “conversion,” and “converting” refer to the change in macrophage phenotype from one macrophage phenotype to another macrophage phenotype.

The terms “induce,” and “induction” refer to the promoting a change in macrophage phenotype from one macrophage phenotype to another macrophage phenotype.

The terms “isolated,” “purified,” or “biologically pure” refer to material that is free to varying degrees from components which normally accompany it as found in its native state. “Isolated” denotes a degree of separation from original source or surroundings. “Purified” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified. “Purified” can also refer to a molecule separated after a bioconjugation technique from those molecules that were not efficiently conjugated.

The phrase “macrophage conversion” as used herein refers to the sequential change in macrophage phenotype, e.g., a macrophage transitioning from pro-inflammatory (M1) to pro-healing (M2a) to pro-remodeling (M2c) phenotypes.

The term “wound macrophage” as used herein refers to a hybrid population of macrophages in a wound including a spectrum of macrophage phenotypes and subtypes that include, but are not limited to, M0, M1, and M2 (including M2a and M2c) macrophages.

The term “M1 macrophage” as used herein refers to a macrophage phenotype. M1 macrophage are classically activated or exhibit an inflammatory macrophage phenotype.

The term “M2” broadly refers to macrophages that function in constructive processes, like wound healing and tissue repair. Major differences between M2a and M2c macrophages exist in wound healing.

The term “M2a macrophage” as used herein refers to a macrophage subtype of pro-healing macrophages. M2a macrophages are involved in immunoregulation.

The term “M2c macrophage” as used herein refers to a macrophage subtype of pro-remodeling macrophages. M2c macrophages are involved in matrix and vascular remodeling and tissue repair.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

As used herein, the term “ratio” refers to a relationship between two numbers (e.g., scores, summations, and the like). Although, ratios can be expressed in a particular order (e.g., a to b or a:b), one of ordinary skill in the art will recognize that the underlying relationship between the numbers can be expressed in any order without losing the significance of the underlying relationship, although observation and correlation of trends based on the ration may need to be reversed. For example, if the values of a over time are (4, 10) and the values of b over time are (2, 4), the ratio a:b will equal (2, 2.5), while the ratio b:a will be (0.5, 0.4). Although the values of a and b are the same in both ratios, the ratios a:b and b:a are inverse and increase and decrease, respectively, over the time period.

As used herein, the term “initial medical encounter” encompasses one or more related interactions with one or more medical professionals. For example, if a subject visits her primary care provider's office regarding a wound, her interactions with a medical assistant, nurse, physician's assistant, and/or physician would constitute a single “medical encounter.” Likewise, a subject's interactions with a plurality of medical professionals during an emergency department visit would also constitute an “initial medical encounter.” The term “initial medical encounter” also encompasses the first interaction with a medical professional specializing in wound care. For example, a subject's first appointment with a wound clinic could be considered an “initial medical encounter.” The “initial medical encounter” can be the actual first or subsequent encounter with a medical professional. For example, a medical professional may not obtain a first sample until after the wound persists from a first appointment to a second appointment.

As used herein, the term “sample” includes biological samples of materials such as organs, tissues, cells, fluids, and the like. In one embodiment, the sample can be obtained from a wound. In other embodiments, the sample can be obtained from inflamed tissue such as tissue afflicted with Inflammatory Bowel Syndrome, Crohn's disease, and the like. In still another embodiment, the tissue can be cancerous tissue (in which an increase in M1/M2 ratio would be desired for inhibition of tumor progression and a low or decreasing M1/M2 ratio would be indicative of tumor progression and metastasis). In still another embodiment, the sample can be obtained from an in vivo or in vitro testing platform such as a culture dish, a scaffold, an artificial organ, a laboratory animal, and the like.

As used herein, the term “wound” includes injuries in which the skin (particularly, the dermis) is torn, cut, or punctured. Examples of types of wounds that can be assessed using embodiments of the invention described herein include external wounds, internal wounds, clean wounds (e.g., those made in the course of a medical procedure such as surgery), contaminated wounds, infected wounds, colonized wounds, incisions, lacerations, abrasions, avulsions, puncture wounds, penetration wounds, gunshot wounds, and the like. Specific wound examples include diabetic ulcers, pressure ulcers (also known as decubitus ulcers or bedsores), chronic venous ulcers, burns, and medical implant insertion points. Embodiments of the invention are particularly useful in identifying nonhealing wounds that are prevalent in diabetic and/or elderly subjects.

### DETAILED DESCRIPTION OF THE INVENTION

Previously proposed indicators of healing outcome biomarkers for diagnosis of nonhealing wounds suffer from high variability between wounds, technical difficulties in detection methods, and impose burdens both on the patient and the care provider because the methods of detection are not a normal part of the wound care regimen.

Aspects of the invention utilize genetic information about macrophage behavior to identify differences between healing and nonhealing in diabetic chronic wounds. Macrophages are the central cell of the inflammatory response and are recognized as primary regulators of wound healing, with their phenotype orchestrating events specific to the stage of repair. Macrophages exist on a spectrum of phenotypes ranging from pro-inflammatory or “M1” to anti-inflammatory and pro-healing or “M2.” M2 macrophages can be further categorized as M2a, M2b, or M2c macrophages. In early stages of wound healing (1-3 days), M1 macrophages secrete pro-inflammatory cytokines and clear the wound of debris. In later stages (4-7 days), macrophages switch to the M2 phenotype and promote extracellular matrix (ECM) synthesis, matrix remodeling, and tissue repair. If the M1-to-M2 transition is disrupted, depicted by persistent numbers of M1 macrophages, the wound suffers from chronic inflammation and impaired healing.

While abnormal macrophage activation in diabetic wounds has been thoroughly described in animal models of diabetes, it has not yet been assessed in human diabetic wounds.

Applicant proposes that absolute, relative, and proportional counts of M1, M2, M2a, M2b, and/or M2c macrophages as well as surrogates thereof can be utilized to predict whether a wound will heal.

In one embodiment of the invention, Applicant investigated differential expression of M1 and M2 genes over time in human diabetic wounds, hypothesizing that healing wounds would exhibit a decrease in the relative proportion of M1 to M2 macrophages. Furthermore, Applicant investigated if gene expression signatures of M1 and M2 macrophages cultured in vitro could be used to quantify wound healing progression, and found that this method may hold potential as a novel noninvasive or minimally invasive diagnostic assay.

Methods of Assessing a Sample and/or Predicting Whether a Wound Will Heal

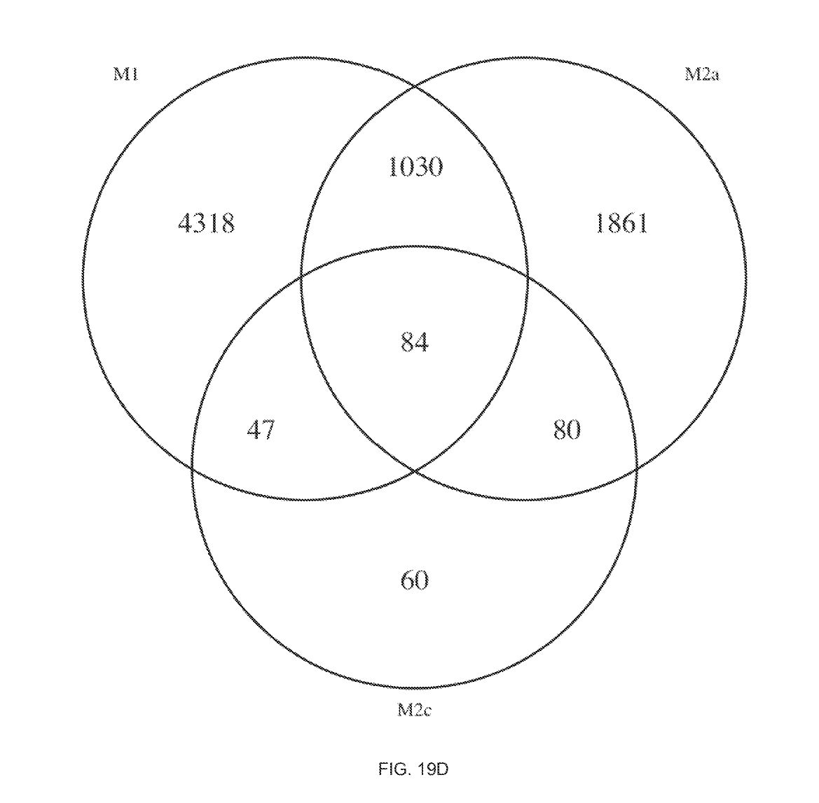

Referring now to FIG. 18, a method 1800 of predicting whether a wound will heal according to an embodiment of the invention is depicted.

In step S1802, a first measurement of a first macrophage phenotype population within a first sample obtained from a wound is obtained.

Exemplary techniques for obtaining a sample from a wound are discussed herein.

The first measurement of a first macrophage phenotype population can be any measurement of the number of macrophages within a sample or a volumetric or mass unit thereof or a surrogate for the same. For example, the number of macrophages can be measured using microscopy or one or more measurements correlated with a population of macrophages can be measured using one or more techniques that measure the amount of a substance produced or expressed by the population of macrophages.

Suitable techniques for measuring a surrogate of macrophage population include, but are not limited to, flow cytometry, immunostaining, and other techniques for measuring gene expression, protein expression, cytokines, and/or other metabolomics byproducts associated with particular macrophage phenotypes.

Gene expression data can be processed or analyzed using a sets of individual expression values as discuss herein (e.g., through linear sums and other algorithms). Additionally or alternatively, gene expression data can be presented using a variety of gene set enrichment analysis algorithms that assess activation of a family of genes that are associated with a biological pathway or functionality (often referred to as a “gene set”), as opposed to individual genes. Exemplary gene set enrichment analysis algorithms include but are not limited to the GSEA method as described in A. Subramanian et al., “Gene set enrichment analysis: A knowledge-based approach for interpreting genome-wide expression profiles,” 102(43) PNAS 15545-50 (2005) and V. Mootha et al., “PGC-1alpha-responsive genes involved in oxidative phosphorylation are coordinately downregulated in human diabetes,” 34 Nature Genetics 267-73 (2003) and the QUSAGE method as described in G. Yaari et al., “Quantitative set analysis for gene expression: a method to quantify gene set differential expression including gene-gene correlations,” 41(18) Nucleic Acids Res. e170 (October 2013) and available at http://clip.med.yale.edu/qusage/. The GSEA and QUSAGE methods both yield a score that can be used by itself or in a ratio with scores reflective of other macrophage populations to make the comparisons discussed herein.

Various techniques can be utilized to determine which data (e.g., genes and corresponding functions combining particular genes) are particularly relevant in assessing a macrophage population. These techniques can be divided into two broad categories. The first category includes methods that preserve all features (in this case, genes) and may or may not include weighting strategies to give more weight to more important features or based on the correlation of a feature with a certain outcome. For example, statistical hypothesis testing such as a t-test can be used to weight features as described herein, or correlation coefficient of a feature with a certain outcome can be used to weight features. The second category includes methods that use a subset of features. This subset can be obtained through a variety of methods known as dimensionality reduction methods. Dimensionality reduction methods can be either linear such as principal component analysis (PCA), independent component analysis (ICA), singular value decomposition (SVD), and non-negative matrix factorization, or non-linear such as kernel PCA and graph-based methods (also known as Laplacian eigenmaps). The new combinatorial features, which number far less than the number of features all together, are then treated as new variables. Alternative methods for feature subset selection include use of discrimination properties of features. In this regard, if features are treated individually, a variety of class separablility measures such as the receiver operating characteristics (ROC) curves, Fisher's discriminant ratio, and one-dimensional divergence can be used to select a subset of features. These methods, however, do not take into account the correlation that may exist among features and as a result their influence on the classification capabilities of the selected subset of features. To address this limitation, techniques measuring classification capabilities of feature vectors are applied. Neural networks can also be applied for feature generation and selection.

In step S1804, a second measurement of second macrophage phenotype population from the wound is obtained. The second measurement of the second macrophage phenotype population can be a different macrophage phenotype obtained from the first sample or the same macrophage phenotype obtained from a second, later sample from the wound. For example, if a single sample is used, the first measurement can relate to the M1 macrophage population and the second measurement can relate to the M2 macrophage population (e.g., all M2 macrophages or one or more of M2a, M2b, and/or M2c macrophages). If the second measurement is obtained from a second, chronologically later sample from the wound, the first and the second measurement can relate to the same macrophage phenotype in both measurements (e.g., a first measurement of M1 macrophages and a second measurement of M1 macrophages, a first measurement of M2 macrophages and a second measurement of M2 macrophages, a first measurement of M2a macrophages and a second measurement of M2a macrophages, a first measurement of M2b macrophages and a second measurement of M2b macrophages, a first measurement of M2c macrophages and a second measurement of M2c macrophages, and the like, including ratios of measurements).

In step S1806, the first measurement is compared to the second measurement. In one embodiment, this comparison is expressed as a ratio as discussed herein.

In step S1808, a prediction of whether the wound will heal is made based on a result of the comparing step. Without being bound by theory, it is believed that ratios exceeding the thresholds specified in Tables 1 and 2 herein are indicative of wounds that will heal without the need for enhanced techniques such as the use of synthetic skin substitutes, hyperbaric oxygen therapy, or negative-pressure wound therapy.

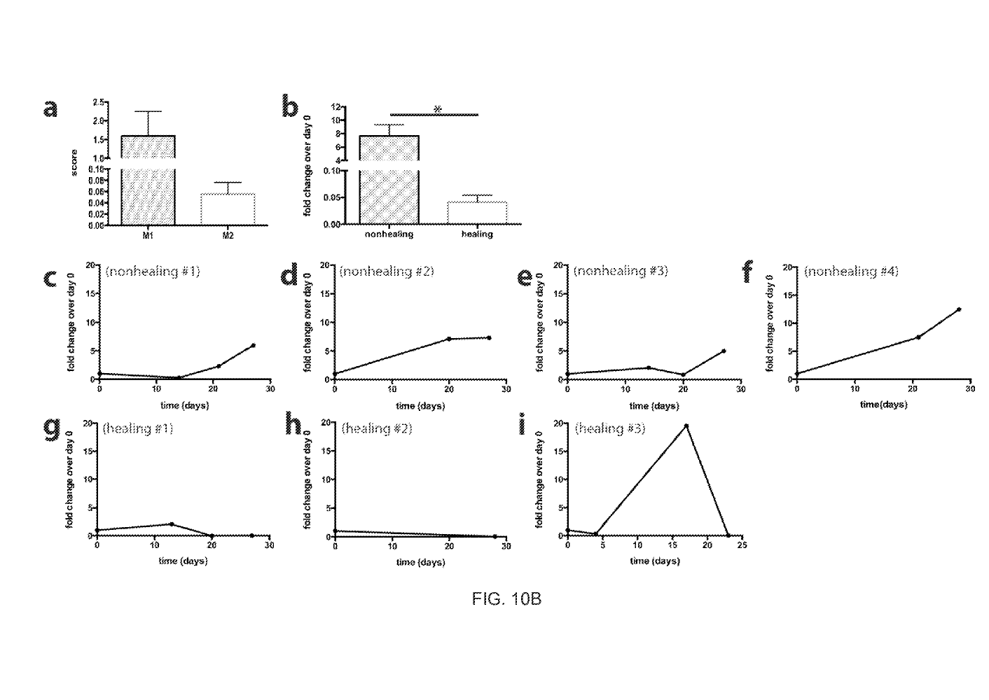

TABLE 1
Exemplary thresholds for wound healing predictions based on single
sample, where the single sample constitutes the genes and methods
described in FIG. 9 (“linear sum method” for M1/M2a and
IL1B/CD163 for M1/M2c)

| First Measurement | Second Measurement | Threshold (1st:2nd) |
|---|---|---|
| M1(t₀) | M2a(t₀) | 320 |
| M1(t₀) | M2c(t₀) | 126 |

TABLE 2
Exemplary thresholds for wound healing predictions based on two samples

| First Measurement | Second Measurement | Threshold (1st:2nd) |
|---|---|---|
| M1(t₀)/M2a(t₀) | M1(t₁)/M2a(t₁) | 4.6 |
| M2a(t₀)/M2c(t₀) | M2a(t₁)/M2c(t₁) | 4 |

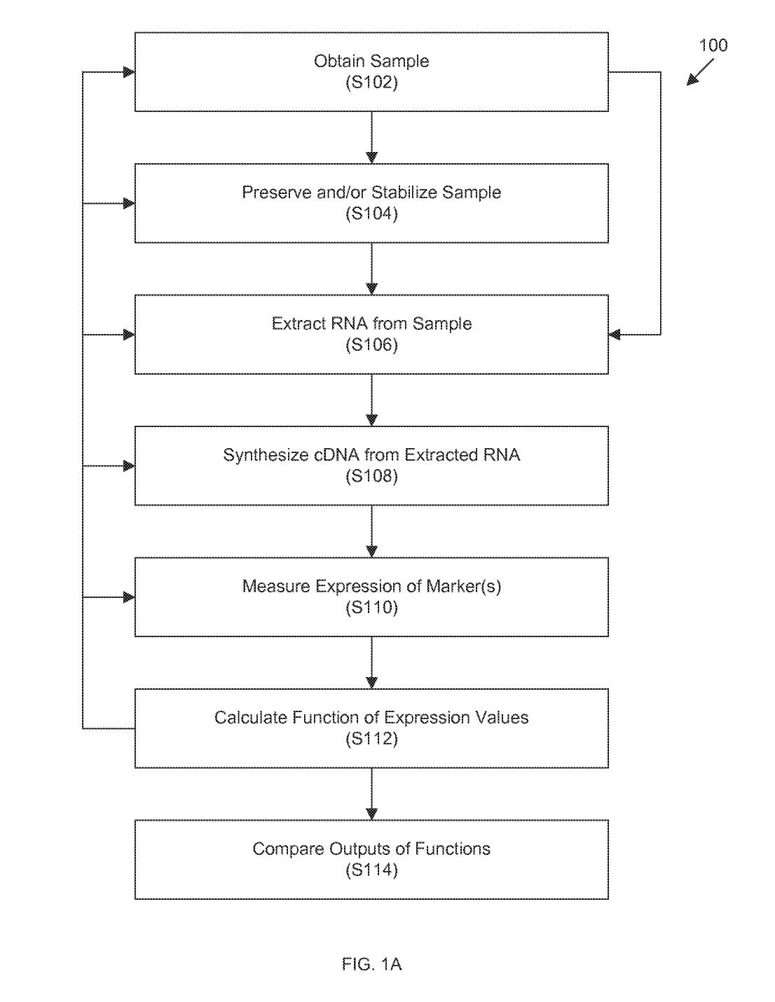

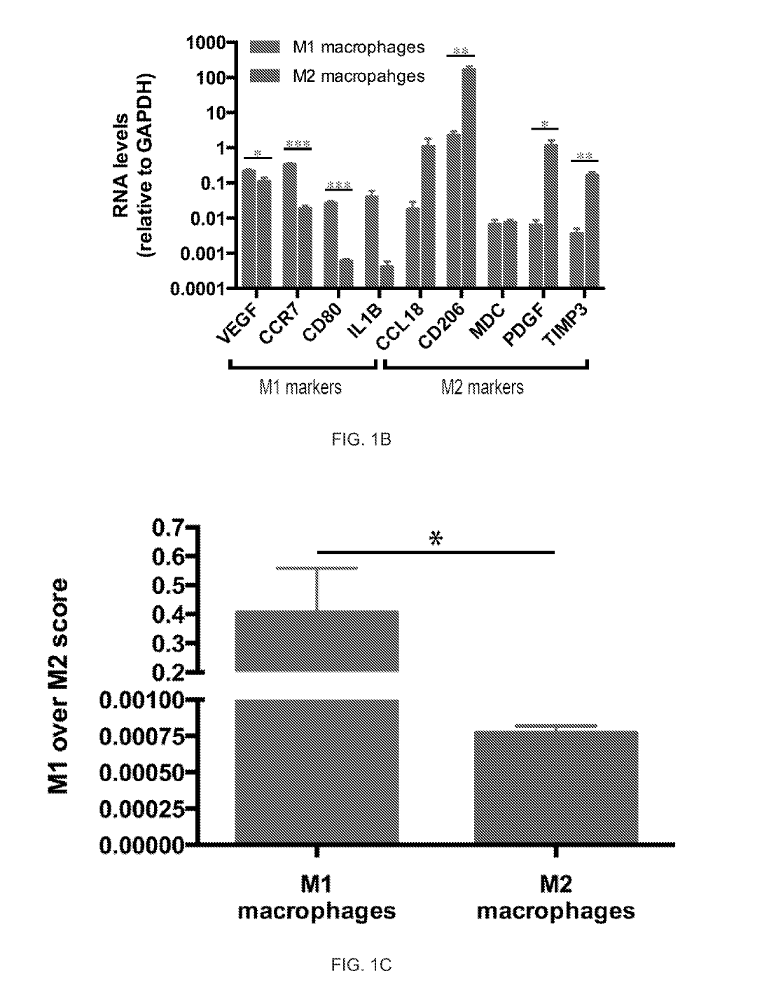

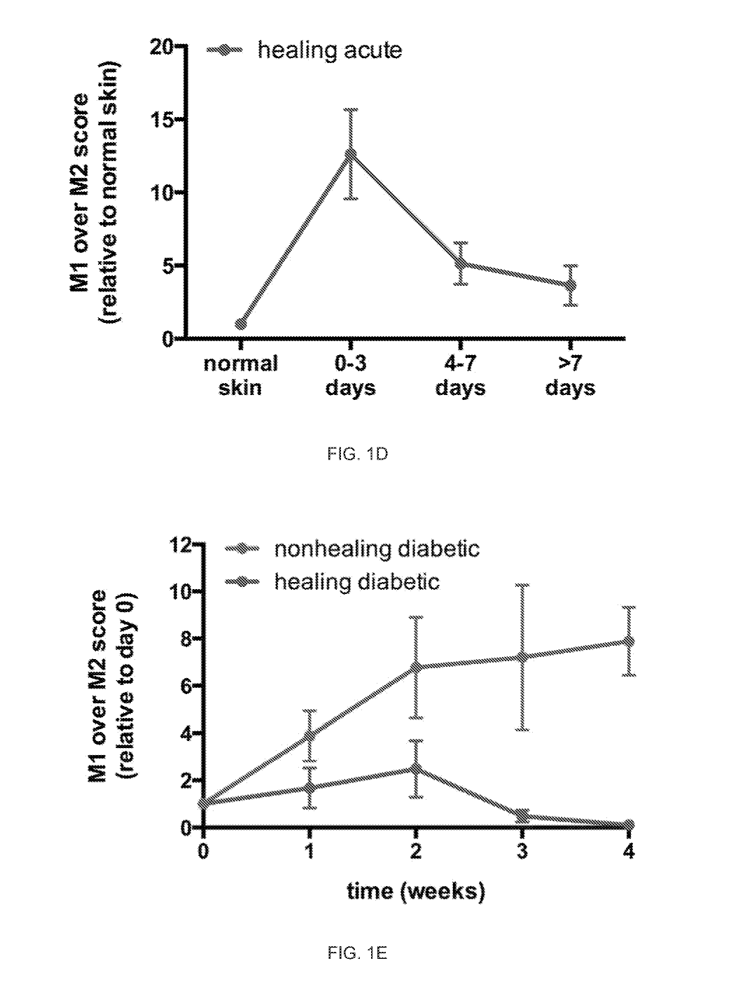

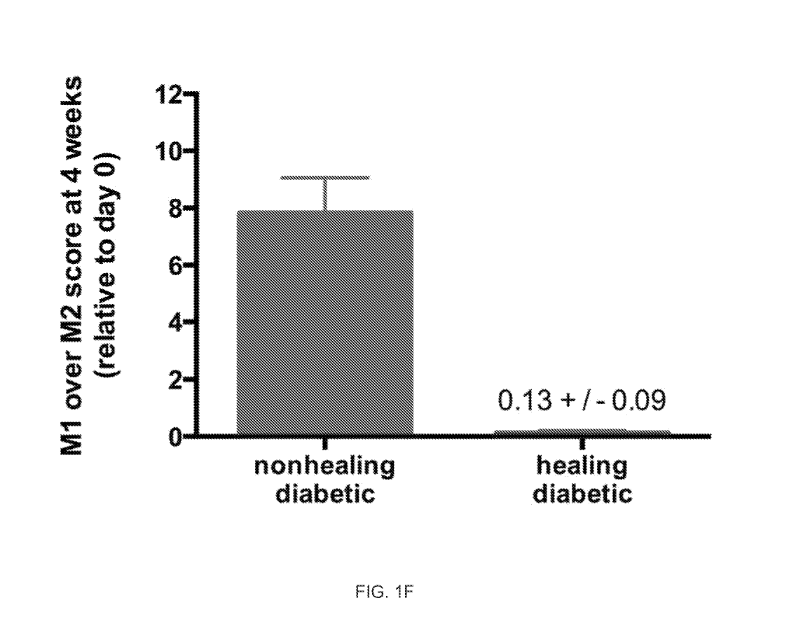

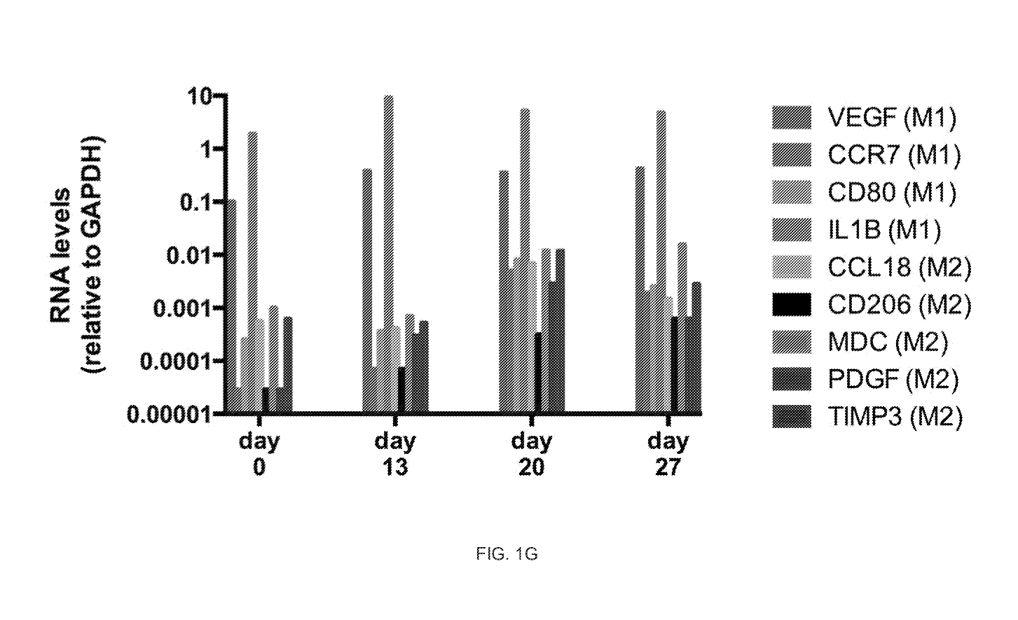

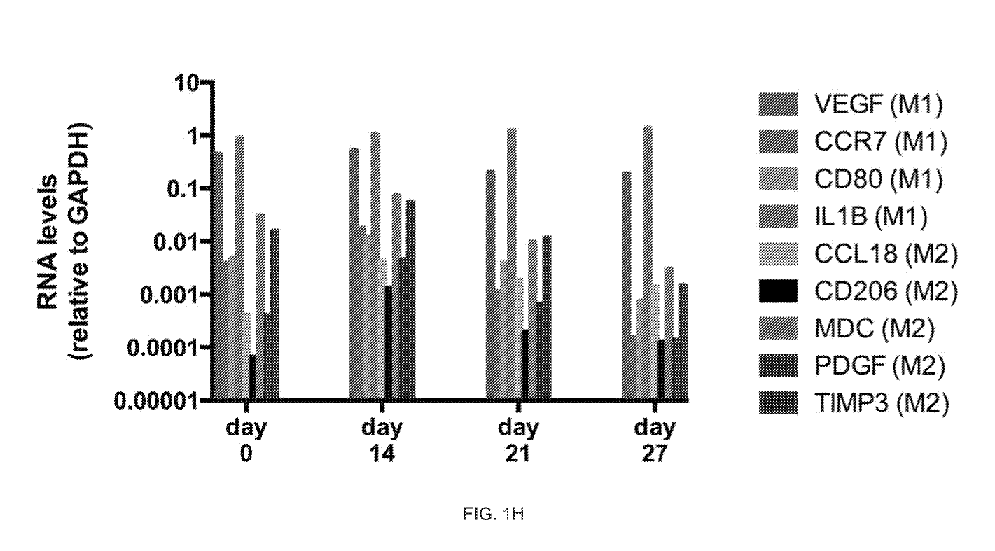

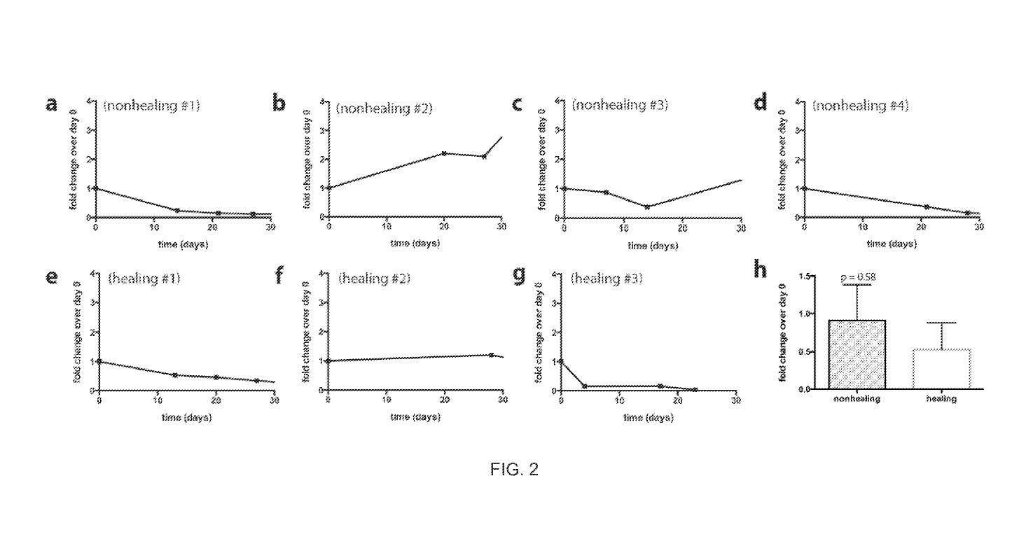

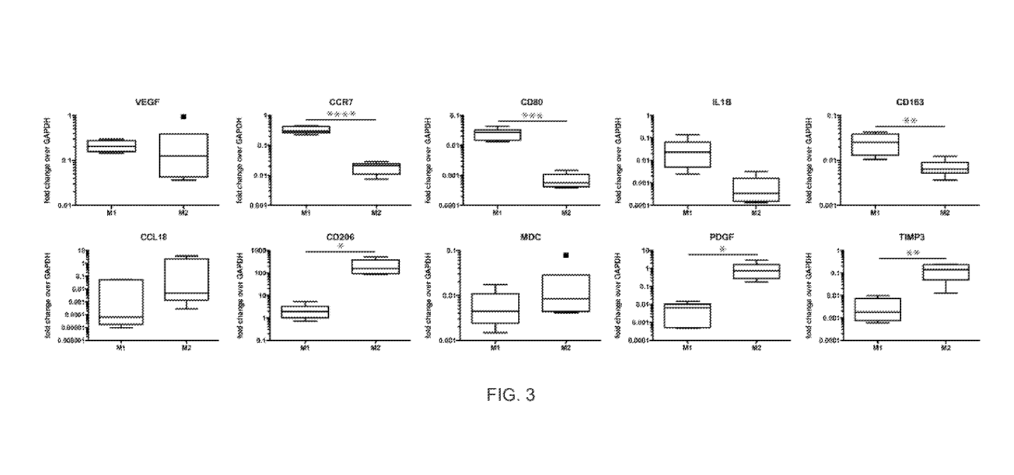

Referring now to FIG. 1A, a method 100 of assessing a sample is depicted.

In step S102, a biological sample can be obtained (e.g., from a wound of a subject). In one embodiment, the biological sample is debrided tissue, which can include, but is not limited to, dead, damaged, or infected tissue. A variety of debriding techniques can be applied.

In one embodiment, mechanical debridement is used in which removal of a dressing from a wound that proceeded from moist to dry will non-selectively remove tissue adjacent to the dressing. This removed tissue can then be separated from the dressing (e.g., by scraping, rinsing, and the like). Advantageously, harvesting of debrided tissue from removed dressings avoids the challenges associated with more invasive approaches and provides sufficient quantities of human wound tissues for quantitative analyses of the cellular content using tissue that would otherwise be discarded.

In another embodiment, surgical debridement can be performed using various surgical tools such as a scalpel, a laser, and the like. Advantageously, harvesting of debrided tissue avoids the challenges associated with more invasive approaches such as using punch biopsies while providing sufficient quantities of human wound tissues for quantitative analyses of the cellular content using tissue that would otherwise be discarded. Although relatively non-invasive procedures can be used, the samples used herein can also be obtained through invasive procedures such as punch biopsies, shave biopsies, incisional biopsies, excisional biopsies, curettage biopsies, saucerization biopsies, fine needle aspiration, and. the like.

In step S104, the sample can be preserved and/or stabilized until further analysis can be performed. For example, the sample can be immersed in a stabilization reagent such as RNALATER® stabilization reagent available from QIAGEN of Venlo, Netherlands.

In step S106, RNA can be extracted from the sample, for example by using a lysing agent such as the TRIZOL® Plus RNA Purification Kit available from Life Technologies of Grand Island, N.Y.

In step S108, complementary DNA ((DNA) can be synthesized from the extracted RNA by using, for example, an APPLIED BIOSYSTEMS® High-Capacity cDNA Reverse Transcription Kit available from Life Technologies.

In step S110, expression of one or more markers can be measured, for example, using quantitative polymerase chain reaction (qPCR). Exemplary approaches to steps S108 and S110 are described in K. L. Spiller et al., “The role of macrophage phenotype in vascularization of tissue engineering scaffolds,” 35(15) Biomaterials 4477-88 (May 2014) (hereinafter “Spiller 2014”).

Exemplary markers associated with M1 macrophage activity include YEGF, CCR7, CD80, and IL1B. Exemplary makers associated with M2 macrophage activity include CCL18, CD206, MDC, PDGF, and TIMP3. Exemplary makers associated with M2c macrophage activity include MMP7, CD163, TIMP1, Marco, VCAN, SH3PXD2B, MMP8, PLAU, PROS1, SRPX2, NAIP, and F5. Sequences for these markers are provided in Tables 3-6 below.

TABLE 3
Sequences for exemplary markers of M1 activity

| Gene | Forward Sequence | Reverse Sequence |
|---|---|---|
| CCR7 | TGAGGTCACGGACGATTACAT (SEQ ID NO: 1) | GTAGGCCCACGAAACAAATGAT (SEQ ID NO: 2) |
| CD80 | AAACTCGCATCTACTGGCAAA (SEQ ID NO: 3) | GGTTCTTGTACTCGGGCCATA (SEQ ID NO: 4) |
| IL1B | ATGATGGCTTATTACAGTGGCAA (SEQ ID NO: 5) | GTCGGAGATTCGTAGCTGGA (SEQ ID NO: 6) |
| (IL1P) | | |

TABLE 4
Sequences for exemplary markers of M2 activity

| Gene | Forward Sequence | Reverse Sequence |
|---|---|---|
| CCL18 | GCTCTCTGCCCGTCTATACC (SEQ ID NO: 7) | GGGCTGGTTTCAGAATAGTCAACT (SEQ ID NO: 8) |
| CD163 | TTTGTCAACTTGAGTCCCTTCAC (SEQ ID NO: 9) | TCCCGCTACACTTGTTTTCAC (SEQ ID NO: 10) |
| CD206 | AAGGCGGTGACCTCACAAG (SEQ ID NO: 11) | AAAGTCCAATTCCTCGATGGTG (SEQ ID NO: 12) |
| (MRC1 | | |
| MDC | GCGTGGTGTTGCTAACCTTCA (SEQ ID NO: 13) | AAGGCCACGGTCATCAGAGT (SEQ ID NO: 14) |
| (CCL22 | | |
| PDGFB | CTCGATCCGCTCCTTTGATGA (SEQ ID NO: 15) | CGTTGGTGCGGTCTATGAG (SEQ ID NO: 16) |
| TIMP3 | ACCGAGGCTTCACCAAGATG (SEQ ID NO: 17) | CATCATAGACGCGACCTGTCA (SEQ ID NO: 18) |

TABLE 5
Sequences for exemplary markers of M2a activity

| Gene | Forward Sequence | Reverse Sequence |
|---|---|---|
| MDC | GCGTGGTGTTGCTAACCTTCA (SEQ ID NO: 19) | AAGGCCACGGTCATCAGAGT (SEQ ID NO: 20) |
| (CCL22) | | |
| CD206 | AAGGCGGTGACCTCACAAG (SEQ ID NO: 21) | AAAGTCCAATTCCTCGATGGTG (SEQ ID NO: 22) |
| (MRC1) | | |

TABLE 6
Sequences for exemplary markers of M2c activity

| Gene | Forward Sequence | Reverse Sequence |
|---|---|---|
| CD163 | TTTGTCAACTTGAGTCCCTTCAC (SEQ ID NO: 23) | TCCCGCTACACTTGTTTTCAC (SEQ ID NO: 24) |

Lists of the most expressed genes for M1, M2a, and M2c macrophage populations are provided in Tables 7, 8, and 9, respectively. The genes are arranged in descending order by rows and then by columns. HUGO Gene Nomenclature Committee (HGNC) symbols are provided for each gene. Corresponding ENSEMBL IDs and EntrezGene IDs are provided in the files incorporated by reference herein and are also available through publicly available databases.

TABLE 7
Most expressed genes for M1 macrophages

| | | | | |
|---|---|---|---|---|
| B2M | C6orf48 | CPEB3 | C1RL-AS1 | RPL7P18 |
| HLA-B | RSAD2 | C22orf46 | EBI3 | PPP1R17 |
| HLA-A | ZC3H12A | OASL | ZNF165 | MORN3 |
| SOD2 | C1RL | RASGRP1 | APOBEC3B | ADORA2A |
| HLA-C | H1F0 | ZFAND4 | CCL8 | GPRC5B |
| HLA-E | PSMB10 | C21orf91 | NKG7 | DSCAML1 |
| WARS | PSMB9 | STAT4 | NEURL3 | MT-TM |
| SAT1 | TNIP2 | PDE4D | LAMA5 | CXCL10 |
| CD74 | LMTK2 | MICA | MFSD4 | CKMT1B |
| RNF213 | MT1G | IL15RA | A4GALT | CTRL |
| PLAUR | SERTAD1 | LAMA3 | IGFLR1 | PLLP |
| ACSL1 | TRAF3IP2 | GADD45G | MMP25 | ASAP3 |
| NCOA4 | NFKBIZ | IL15 | HOXB2 | PLIN5 |
| MCL1 | PSTPIP2 | WEE1 | SPOCK2 | UPK3A |
| SNX10 | HLA-H | CLEC2D | ADAM19 | NTNG2 |
| CYP27B1 | C1QC | MDGA1 | HAPLN3 | MTND1P11 |
| MMP14 | PELI1 | ERO1LB | TWF1P1 | NFE4 |
| STAT1 | TRIM21 | VAMP5 | HOXA1 | CES1P1 |
| HLA-DRA | XAF1 | TUBE1 | MIR4519 | OR2A7 |
| CFLAR | DYNLT1 | RAB43 | ITGA9 | GK-IT1 |
| TNFAIP2 | MSRB1 | CMKLR1 | IFITM1 | SBK3 |
| SLAMF7 | ADAM28 | GVINP1 | FBLIM1 | SRD5A3-AS1 |
| GNA13 | OTUD1 | RMI2 | TSPAN5 | PROB1 |
| CD83 | APOO | C4orf32 | SH2D3A | KCNE1L |
| BTG1 | TMEM170A | ITK | ANKRD22 | EXOC3L1 |
| LAP3 | SEMA4A | WWC3 | CLIC3 | RAB44 |
| IFI6 | BTN3A3 | MAMDC4 | LNX1 | TNK2-AS1 |
| ZNFX1 | MYO1G | TMOD2 | RAPSN | KCNN1 |
| IL8 | TOR1B | FAM26F | MAGI2-AS3 | HLA-DRB9 |
| ATF3 | DPP4 | ZNF702P | LMTK3 | C9orf50 |
| TXN | FBXO32 | CHST3 | GATA2 | BAIAP2L1 |
| PTPRJ | PVR | CCDC149 | MEFV | TMEM54 |
| PARP14 | HIVEP2 | IRF9 | NFIX | TINCR |
| TAP1 | IFIT2 | DFNB31 | MT1L | GBP7 |
| ICAM1 | PRPF3 | OSM | UNC13A | SCG3 |
| TYMP | APOL1 | OSBP2 | SCN4B | DMGDH |
| NFE2L1 | APOL3 | GCH1 | PTGES3P1 | NKX3-1 |
| CLCN7 | ARL5B | HLA-DRB6 | ARHGEF35 | LAX1 |
| SCPEP1 | RELB | CRISPLD2 | IGFBP4 | PRPH |
| MTHFD2 | ST8SIA4 | PLEKHG3 | DNAJC3-AS1 | KRT7 |
| NFKBIA | N4BP2L1 | MAP1LC3A | KIAA1045 | TRPA1 |
| STOM | RGL1 | DTNB | GBP1P1 | RAB39B |
| STAT2 | FCHSD1 | NCF1C | NOTCH4 | CFH |
| PIM1 | CCL5 | KSR1 | PLEKHN1 | C11orf96 |
| HLA-DPA1 | ENDOD1 | CFB | FAM46C | IL1RL1 |
| KYNU | MT1E | LYSMD2 | IL32 | HLA-V |
| TSC22D1 | PPA1 | MIR155HG | BEGAIN | LPAR4 |
| NAMPT | TDRD7 | MEIS3 | KCNA3 | ANXA3 |
| KIAA0247 | PLA2G4C | RTP4 | DOCK9 | HSD17B13 |
| HLA-DRB1 | PHF1 | RAB3IP | IL27 | PBX1 |
| NINJ1 | C19orf12 | RARG | GRHL1 | S100A3 |
| PTAFR | FADS3 | HCAR3 | FAM71A | DTX3 |
| PNRC1 | SLC37A1 | EDN1 | TFAP2E | DMBX1 |
| C15orf48 | DHX58 | BBS12 | TXNP6 | LINC00482 |
| IL13RA1 | GPR157 | CLEC4E | HIP1R | MST1R |
| TNFAIP3 | RHBDD2 | IRF6 | CD6 | RGS11 |
| B4GALT5 | TAPBPL | STAP2 | HLA-DQB2 | FAM71E1 |
| APOL6 | TAP2 | BATF2 | CCDC80 | PRSS8 |
| C3 | AP1AR | TNF | CAV2 | SOX5 |
| PILRA | ACVRL1 | DIRAS2 | PNPLA1 | RPL32P1 |
| SLC31A2 | NAB1 | ITGA2 | AOC2 | RGS9 |
| PSME2 | IFI35 | RARRES3 | GFPT2 | ICOS |
| GBP2 | USP11 | CEBPE | VWA7 | SERPINB7 |
| CD48 | HMCES | CCDC154 | BEAN1 | KIT |
| RDX | HLA-DQA1 | CXCL1 | CXCL9 | CPA4 |
| ZCCHC6 | SEMA4D | ASPHD2 | GALNT3 | ARSI |
| MT2A | PROCR | PERP | P2RY10 | CCL15 |
| LILRA3 | NUPR1 | PARD6B | FAM177B | ABCC11 |
| RHBDF2 | RUNX3 | VMO1 | KL | FAAH2 |
| SMAP2 | TIFA | CD38 | CCL20 | C9orf172 |
| TBC1D9 | INSIG2 | AMZ1 | ANKRD1 | EXOC3L4 |
| CXCL5 | TBKBP1 | PIPOX | CLEC6A | EPN3 |
| ALDH2 | MAP3K8 | SPAG1 | HDC | MTND6P5 |
| CD274 | ULK2 | ZEB1 | PRRG2 | PRF1 |
| UBE2L6 | ZMYND15 | TSHZ3 | RHEBL1 | FGF2 |
| METTL9 | GIMAP2 | BRIP1 | PRDM8 | CCDC73 |
| DUSP5 | IDO1 | TMEM154 | LINC00189 | SAPCD1-AS1 |
| B4GALT1 | BCL9L | LRRC32 | PTPRH | NT5C3AP1 |
| TANK | MESDC1 | VILL | UPB1 | ORM2 |
| DOCK4 | ARNTL2 | GRAMD3 | TMEM25 | TMEM92 |
| SDC4 | IL1B | HLA-DOA | UPK3B | FAM3B |
| RAP1B | LAMB3 | CREB5 | CEACAM4 | TSPAN1 |
| LY6E | CSRNP2 | HCAR2 | SPAG5-AS1 | NBEAP1 |
| HLA-F | PSMA6 | ARHGEF5 | PLEKHA7 | SMIM5 |
| CDC42SE2 | PDP1 | DDR1 | C15orf62 | NRG2 |
| SLFN5 | DTX2 | STOML1 | LINC00996 | SPTA1 |
| ADAMDEC1 | ASAP2 | NCF1B | SKOR1 | CYP4F3 |
| TMEM140 | MICB | AMER1 | RND1 | CTLA4 |
| PSME1 | LONRF1 | PGAP1 | ACTRT3 | TREML4 |
| SNHG16 | IFIT5 | KLF5 | GP1BA | SERINC4 |
| SLC39A8 | NMI | TLCD2 | MAP3K9 | MTHFD2P7 |
| ARID5B | FBXO6 | CARD16 | FCGR1C | KRT23 |
| CSF2RB | GADD45B | RNF207 | CCDC157 | ITM2A |
| DTX3L | MDK | REC8 | HES4 | C19orf84 |
| PCNX | CREM | THNSL2 | SIK3-IT1 | CCL19 |
| TRIM25 | DDX58 | PDGFRB | IL6 | CCR10 |
| SAMD9L | LAMP3 | TNFRSF18 | FGF13 | FEZ1 |
| OPTN | SCO2 | USP18 | ANKRD33B | GPR174 |
| BAZ2A | PLEKHM3 | SEMA4C | F2RL1 | RDH16 |
| XRN1 | SLC2A6 | SERPINI1 | FAM227B | IL22RA1 |
| INHBA | PMAIP1 | C12orf79 | ALG1L13P | ELOVL3 |
| MET | FAM135A | PTCRA | OR2A1-AS1 | DMC1 |
| APOL2 | FAS | ABCC6 | TNIP3 | TTC22 |
| SOCS3 | C9orf91 | IGF2BP3 | MYCBPAP | LINC00337 |
| LGALS3BP | MEI1 | TMEM216 | NKAPL | SMCO2 |
| PVRL2 | SASH1 | ZG16B | EYA4 | RNU7-45P |
| TRIM22 | TMEM150B | MAMLD1 | LYPD3 | C6orf100 |
| MKNK2 | IL18BP | TMEM229B | TRIM31 | FAM169A |
| WTAP | MPZL3 | ABTB2 | RLTPR | HMGB1P3 |
| ACOT9 | ITPR3 | ASCL2 | MUC1 | MAPK8IP2 |
| RIPK2 | PLAGL2 | C5orf56 | HLA-DOB | IL12B |
| SAMD9 | NCF1 | ZNF462 | SIX5 | ADRB1 |
| ZBTB38 | LILRB2 | APOBEC3F | SH3BP4 | ANKRD2 |
| AARS | SMPD2 | SLC51B | ILDR1 | ACHE |
| SBNO2 | IRF7 | RNF144A | OLIG2 | SPAG6 |
| P2RX7 | FOXO4 | PARP15 | CYP4F22 | TRIM55 |
| P2RX4 | VPS9D1 | STX1A | TMPRSS4 | OR2A20P |
| CD40 | JMY | GLIS3 | GCNT4 | CDC42BPG |
| NLRC5 | NT5C3A | NAGS | ATP8A2 | PADI4 |
| ANKRD13A | APOBEC3G | RPS6KA5 | APOBEC3H | CEACAM1 |
| ERAP2 | PPP3CC | LRRCC1 | BANK1 | CAMK1G |
| TLR2 | C1orf132 | SDCBP2 | SRSF12 | TULP2 |
| RNF144B | TBX21 | TPT1-AS1 | ADAMTSL4-AS1 | MIR3945 |
| NBN | NOD2 | CBLN3 | CEACAM19 | DNAJC22 |
| IFNLR1 | CCR7 | FZD4 | CCR3 | MT1A |
| RICTOR | GBP4 | HLA-K | BAIAP3 | GFAP |
| C1QA | CCNA1 | KCNG1 | ITPKA | RPL21P44 |
| GPBP1 | GBP3 | S100P | IGHEP2 | LIPH |
| CALCOCO2 | HIP1 | NRCAM | IL31RA | AJUBA |
| TMEM38B | ISG15 | XIRP1 | CIB2 | ISLR |
| CUL1 | SIGLEC1 | IL12RB1 | CCDC96 | CPXM1 |
| CLU | ISOC1 | SLC2A12 | TMEM8B | RNF43 |
| LSS | CBX4 | PTGES | EXPH5 | DRD4 |
| GBP1 | RHOU | PTK7 | PLA2G16 | EBF4 |
| LCP2 | SLC25A28 | FSTL1 | HESX1 | GREB1L |
| NUB1 | MOB3C | TAF1A | PAX5 | NELL2 |
| MXD1 | PARP3 | YES1 | ADAMTS2 | RSPO3 |
| RNF114 | GIMAP6 | HMGN2P46 | SLC38A5 | B3GNT3 |
| AP5B1 | MLKL | FBN1 | SPRY4 | HCG4P11 |
| IRF1 | C15orf39 | C6orf223 | SYNPO2 | SNAP25 |
| ECE1 | FAM177A1 | CASZ1 | MT-TW | CYP2E1 |
| MARCKS | OVOL1 | TUBD1 | AGBL2 | IGFBP3 |
| ITGB7 | NFE2L3 | VNN3 | HCG4P7 | SLC8A2 |
| PPP4R2 | EREG | RRAD | CNTNAP1 | RN7SL124P |
| HLA-DPB1 | KREMEN1 | FAM225A | IDO2 | TCEA3 |
| BTN3A1 | PTGS2 | ATHL1 | MN1 | PLXNA4 |
| BIRC3 | GOLM1 | IKZF4 | SPATC1 | LINC01093 |
| PARP9 | ITPKC | PSME2P2 | SYNGR3 | KCTD14 |
| LRRK2 | C3AR1 | GRIN3A | CACTIN-AS1 | CXCR3 |
| CCSER2 | IFI44 | CEACAM21 | DNAJC19P5 | SLC12A3 |
| STX11 | PRRG4 | AFAP1 | FBXO2 | NNMT |
| SAMD8 | CSF2RA | IFI44L | TEAD4 | SEZ6L |
| RAPGEF2 | CIITA | CEP19 | EPB41L5 | CTHRC1 |
| OAS3 | DGAT2 | RHOH | LYPD5 | PDE9A |
| FEM1C | HEBP2 | MEP1A | CA13 | BCL6B |
| OSGIN2 | C2 | SGPP2 | LAP3P2 | IL36G |
| JAK2 | CXCL2 | APOL4 | TMEM110-MUSTN1 | ALMS1P |
| ITPRIP | SLC25A37 | FAM47E-STBD1 | EPHX3 | C15orf26 |
| ELMO2 | RINL | HLA-DQA2 | ORM1 | LINC00243 |
| NUP50 | APBA3 | SYT12 | C4A | GBP6 |
| TRANK1 | TRIM5 | CD80 | EPHA2 | VSIG2 |
| CNP | MB21D1 | GPBAR1 | TECTA | PPP1R1A |
| KDM7A | HSBP1L1 | GPR133 | NTN1 | LINC00158 |
| CREBRF | LRRC61 | GIMAP7 | CR1L | CAND2 |
| DAG1 | BPGM | N4BP3 | DKK2 | ABCC6P1 |
| RCN1 | GIMAP8 | ARHGEF34P | TSPAN9 | KLF15 |
| RILPL2 | PRKAR2B | SHISA4 | RN7SL834P | SPNS2 |
| KLHL21 | LMNB1 | ZNF425 | HIC1 | OR7E140P |
| ATG2A | PIM2 | NID1 | IRG1 | FDPSP3 |
| VEGFA | TAGAP | TJP3 | PLA1A | FAT3 |
| IFIH1 | ETS2 | FAAH | BACH2 | MS4A2 |
| RAP2C | SIAH1 | JMJD7-PLA2G4B | SGSM1 | PRRT2 |
| TGIF1 | ITGA7 | TNFRSF8 | RN7SL473P | KLC3 |
| TRAFD1 | LSR | HCG4P5 | ETV7 | GRIN1 |
| MSC | ST6GALNAC2 | CDC42EP2 | MYO7B | IL17C |
| EIF2AK2 | SIK1 | CD69 | S100A12 | FGF7 |
| CASP4 | CHI3L2 | PCDH12 | CLEC4D | VCAM1 |
| GRAMD1A | TP53INP2 | IL3RA | AURKC | GGT5 |
| RELA | FER1L6 | PKD2L1 | RDH5 | CGNL1 |
| ADM | CXCL3 | TYW1B | CCL1 | INSL3 |
| SLC6A12 | ZSWIM4 | FAM122C | FAM35DP | ADAMTS7 |
| HLA-DQB1 | ZC3H3 | GALNT4 | ARRDC5 | GPR97 |
| RNF24 | KCP | LYSMD1 | TMPRSS9 | HNRNPA1P27 |
| IFIT3 | IRAK2 | ZMYM6NB | RN7SL600P | CDIPT-AS1 |
| CCDC115 | EPS8L2 | GIPR | CD70 | F2RL2 |
| SDS | IFITM3 | KLK4 | LAG3 | ARHGAP40 |
| TNFAIP8 | HERC5 | PTGIR | C1orf61 | CASP5 |
| GBP5 | PLEKHG2 | PSMD6-AS2 | TNK1 | MT-TE |
| C5orf15 | IFITM2 | ADRB2 | CTF1 | KIAA1644 |
| TBK1 | MT1H | KLHDC7B | SLFN12L | RN7SL559P |
| TMEM41B | CMPK2 | SLC9B2 | BEST4 | STEAP4 |
| DIXDC1 | EPSTI1 | MEIS3P1 | ITIH1 | ARTN |
| G0S2 | FPR2 | APOBEC3A | ELF3 | C17orf66 |
| SESN2 | TNFSF10 | HLA-J | C1QTNF1 | TXNP4 |
| PARP10 | BMP6 | CDKN2D | HTR2B | GZMA |
| ZC3H12C | ABLIM1 | ADM2 | WHAMMP3 | MT1DP |
| MIER1 | GPR132 | HSH2D | NYNRIN | LINC00944 |
| ORAI2 | NMRK1 | FCGR1B | CSF3 | MT1JP |
| DUSP10 | RPLP0P2 | APOBEC3D | AASS | RHCG |
| MX1 | CASP1 | JHDM1D-AS1 | SNX15 | CXCR6 |
| CMTR1 | SLC35E4 | NSUN7 | DNAAF1 | SERPINB13 |
| PML | RALGPS2 | BTBD11 | MYO5B | HHIPL2 |
| SCARF1 | PDE4B | NLGN2 | LINC00426 | GRIP2 |
| TSC22D3 | SMPDL3A | CHAC1 | BLK | VEGFC |
| GYPC | ELOVL7 | ISG20 | UBXN10 | DOK5 |
| SIPA1L1 | BCL3 | SSPN | DPYS | ATP1B2 |
| FAM126B | SP140 | EGR3 | ZBP1 | WNT5A-AS1 |
| CNNM4 | FAM124A | EBF1 | FAM185BP | CCM2L |
| KIAA0040 | ARHGAP24 | C1R | FFAR2 | SPRN |
| TMCC3 | TBL1X | PPIL6 | HPD | HOXA10 |
| CARS | NUAK2 | TMEM158 | MYH11 | ITIH5 |
| CCDC50 | MIR29A | LIPG | PRRX1 | GPR171 |
| BAK1 | MIR29B1 | NAMPTL | GAPDHP14 | KCNG2 |
| ITGB8 | PRKD2 | AIM2 | CDC42EP5 | GAL3ST2 |
| C1QB | STARD10 | CMYA5 | RIMS2 | LTK |
| OCSTAMP | HS3ST3B1 | IFI27 | MCC | LTA |
| PRDM1 | SCYL3 | SLCO5A1 | KLK10 | FAM160A1 |
| SIK3 | CLCF1 | TMC4 | CPA3 | SAA2 |
| DCP1A | BBC3 | EPHA1 | FLT3LG | KRT8P31 |
| CSRNP1 | KCNJ2 | CDKN1C | FOLR1 | WFDC2 |
| COQ10B | CLDN7 | TPBG | TWIST1 | IL12RB2 |
| CA12 | KIF3C | NHS | STRA6 | GJA3 |
| GCC1 | XKR8 | CPT1B | JMJD7 | UNC5C |
| HCP5 | TFPI2 | TESPA1 | ROBO1 | PTCHD3P3 |
| CASP10 | HSPBAP1 | DEGS2 | PRICKLE1 | KCNQ3 |
| RFFL | CCDC88C | IL2RA | LRTM2 | ESRP2 |
| HELZ2 | ANGPTL4 | EMR1 | S100A14 | TARM1 |
| ARID5A | SLC9A7P1 | PIK3R3 | HS3ST3A1 | IL18RAP |
| OAS2 | ABCG1 | TNRC18P1 | BCL2L14 | HCAR1 |
| ERN1 | MARCKSL1 | PRSS22 | C22orf42 | MIR4451 |
| RNF19B | SECTM1 | PDCD1 | HPN | NRN1 |
| CCDC69 | LINC01137 | LAD1 | NPPA-AS1 | KCNJ10 |
| ARID3A | A1BG-AS1 | PLAC8 | MTND4P14 | VWA3B |
| RBCK1 | ASNS | GPR64 | BSPRY | CTAGE8 |
| TMEM176B | ODF3B | TICAM2 | ADC | CXCL11 |
| MX2 | CIDECP | CYB5R2 | ANO7P1 | TMEM171 |
| ENPP4 | CYB561 | TRPC2 | ITIH4 | RORB |
| DDIT4 | FAM65B | BEX2 | UBD | ROR2 |
| DSP | MAP3K7CL | ZDBF2 | NLRC3 | SAA1 |
| RAB12 | MAP3K10 | ANXA2R | ADAM11 | SERPINE3 |
| HLA-DRB5 | MYRF | 41886 | C2orf62 | LINC00322 |
| JADE2 | PIGR | ITGA1 | DND1 | DES |
| GSDMD | SP4 | USP30-AS1 | CD7 | ART5 |
| CP | HERC6 | MARVELD2 | SVEP1 | CRB3 |
| OTUD7B | ZHX2 | PANX2 | CPNE5 | CRABP1 |
| KIAA0226 | LINC-PINT | RCN1P2 | MYO1A | TCF7L1 |
| GIMAP4 | ETS1 | INHBE | HEATR4 | L1TD1 |
| ATXN7 | TRIM9 | AVIL | LINC00528 | SHROOM3 |
| FAM20A | GIMAP5 | GJA5 | ITGA10 | LINC00336 |
| RETSAT | SLC39A14 | CADM4 | OCLN | CSF2 |
| TMEM189 | PPP2R5B | PPAP2A | WEE2-AS1 | CHRNA1 |
| CEBPG | ABTB1 | IL23A | DEFB1 | HMSD |
| TNFSF13B | ARID3B | AXL | RXFP1 | C1QL1 |
| IER3 | MDM1 | SERPINB2 | GPR113 | MKX |
| IGFBP6 | MVB12B | C17orf107 | MTND5P14 | C1orf210 |
| DDX60 | IFIT1 | PKN3 | SLC6A9 | SCARA3 |
| SAMSN1 | IL1A | POU6F1 | FOXP2 | ANKRD33 |
| UXS1 | SUSD3 | C8G | WNT3 | OR6D1P |
| CES1 | USP42 | SEC61A2 | LRRC43 | SLC51A |
| ARMC9 | TRAF3IP3 | ZDHHC23 | KCNMB3 | TMEM212-AS1 |
| GTPBP1 | MTMR11 | MPZL2 | BMX | PLA2G2D |
| SP110 | RAB39A | SNAI1 | CD96 | DMKN |
| DAPP1 | LRWD1 | CLUHP3 | B4GALNT3 | FAM26E |
| ICAM3 | NCAM1 | MT1M | JAG2 | UBXN10-AS1 |
| TMEM194A | WNT5A | GOLGA2P5 | PTPRS | CLIC5 |
| HIVEP1 | GPR84 | AKAP2 | CA15P1 | FBXO39 |
| IFRD1 | MT1F | TMEM255B | TIGD3 | GRB7 |
| CDCP1 | HLA-L | LINC00937 | TNNT2 | MEP1B |
| RFX5 | HELB | DSG2 | P2RY6 | GUCY1B2 |
| ZMIZ2 | TMEM45A | PRLR | FAM187B2P | FUT2 |
| GDF15 | GRASP | SLC9A3R2 | SUSD2 | MTNR1B |
| SERPING1 | SWT1 | CLC | STAP1 | LINC00487 |
| TNFAIP6 | ZNF688 | TTC39A | KCNJ2-AS1 | SLC44A4 |
| ZFP36 | C1S | SCN1B | SEMA3F | C22orf31 |
| PLSCR1 | MYEOV | ERMN | PLA2G4B | SLC35F1 |
| LIMK2 | SMARCD3 | KIAA1211 | ODF3L1 | CCL14 |
| PRMT5 | HLA-G | TNFRSF4 | CD8A | SAA2-SAA4 |
| LATS2 | SNHG15 | CDC14B | C4B | FOXD3 |
| TMEM132A | LAYN | SOCS2 | STK4-AS1 | IFNG |

TABLE 8
Most expressed genes for M2a macrophages

| | | | | |
|---|---|---|---|---|
| CCL22 | TMEM55A | DTNA | CYP7B1 | ST8SIA6 |
| LIPA | FCGR2B | THBS1 | KTN1-AS1 | MACROD2 |
| TGM2 | CHCHD7 | GOLGA8B | TAL1 | VSTM1 |
| MGAT1 | SUOX | PDE6G | NFATC2 | FAM95B1 |
| ANPEP | GPD1 | ZNF620 | SNX32 | HLA-DPB2 |
| ANXA11 | CR1 | SLC6A7 | LCA5L | PLAT |
| QSOX1 | XYLT1 | MAP2K6 | LINC00526 | FAM212B-AS1 |
| PICALM | PDE1B | CCL17 | DNAH7 | LRRC46 |
| SEL1L | NHSL1 | NIPAL1 | TMEM169 | GABRA4 |
| HADHA | GADD45A | CCL23 | BEX1 | KCNK3 |
| H6PD | FHL1 | C2orf71 | ATOH8 | FOXC1 |
| ABCC3 | TNFRSF11A | TTC9 | CHDH | CYYR1 |
| CTSC | TTN-AS1 | C9orf9 | GLI1 | EPPK1 |
| KTN1 | COL7A1 | ENHO | ADAMTS15 | NTRK1 |
| HIPK2 | IL21R | SLC24A4 | CACNB4 | CA5A |
| G6PD | PLA2G4A | CXCR2 | METAP1D | C2orf91 |
| PCM1 | C17orf58 | PTPRF | LRRC1 | ARHGAP26-IT1 |
| TBC1D8 | TRAF5 | TDRKH | HS3ST2 | SIGLEC8 |
| PFKP | CD22 | SYT17 | ROR1 | SEC14L5 |
| SASH3 | ACE | ADORA3 | ANKRD13B | POU3F1 |
| SLC7A8 | MYC | CD1B | ALOX15 | IL22RA2 |
| SLA | AUH | ERRFI1 | NAT8L | SLC9A2 |
| PAM | TNFSF13 | LINC00607 | GUCA1A | RHO |
| MAN2A1 | PTPN4 | ABCC2 | VSTM4 | AP3B2 |
| ADD3 | COL5A3 | SMTNL2 | ESPNL | LINC00484 |
| PTGS1 | NDFIP2 | COL11A2 | FAM198A | HCRTR1 |
| PALLD | QPRT | LHFP | CALD1 | SRL |
| AKIRIN1 | ARL4C | SMARCA1 | PALD1 | SEMG1 |
| AMPD2 | CAMK1D | LINC01160 | CACNA1D | ERVFRD-1 |
| PSME4 | B3GALTL | PRRT3 | SYT6 | GPC6 |
| HSPH1 | ABCG2 | SLC25A15 | OSBPL10 | FST |
| SLC27A3 | MIR4435-1HG | PTPRU | COL4A1 | KIAA1462 |
| GALNT12 | ECHDC3 | EHF | PCSK1 | LINC01114 |
| CD300LB | NUDT16P1 | OLFML3 | PLCL2-AS1 | GRIK1-AS1 |
| EEA1 | CISH | CCDC85C | UCN2 | WDR86-AS1 |
| FLT1 | CCL4 | PBX4 | C17orf64 | CTTNBP2 |
| FPR3 | CLEC4A | TREML1 | EMBP1 | ELFN1 |
| SPOCD1 | PDGFC | LRRC4 | SNCAIP | CCL13 |
| FAR2 | C3orf18 | ZNF827 | BACE2 | CCL26 |
| SPINT2 | MRC1 | CD1C | MIR621 | ZNF705A |
| OSBPL1A | CD1D | CENPV | CH25H | MPL |
| LIMA1 | SLC47A1 | SETBP1 | RORC | CCDC85A |
| LILRB1 | OTUD6B | KIAA1024 | CR2 | ROBO4 |
| C1orf162 | EGLN3 | PLXNA2 | CD1E | FHL2 |
| PIK3R1 | ADAMTSL4 | KIAA1161 | LINC00639 | TENM4 |
| ARHGAP26 | CCDC85B | WDR66 | DUOXA1 | RDH8 |
| FABP4 | PPP1R16B | CLEC10A | LINC00941 | DUOX2 |
| SIGLEC10 | TPTEP1 | MGAT3 | SH3BGRL2 | HSD3BP5 |
| SUCNR1 | C10orf128 | ARHGEF28 | WNT5B | SFRP4 |
| HSPG2 | DDO | RASAL1 | CXCR2P1 | GADL1 |
| NR4A3 | IFFO2 | CELSR2 | TINAGL1 | SLC39A12 |
| PKD2 | NMB | HS3ST1 | LY86-AS1 | RAB3C |
| RCAN1 | PVRL1 | GAS6 | SLC18A2 | MAOB |
| EMB | CCRN4L | CUBN | DNASE1L3 | DTHD1 |
| ETV3 | TMEM130 | ITGA11 | NEO1 | CST2 |
| ACOT7 | GATM | TSPAN12 | UNC80 | ELF5 |
| SNX8 | EGFL7 | MORC4 | ANKEF1 | FAM27A |
| TTC39B | CBR3 | FCER2 | RGMB | FOXD4L1 |
| WFS1 | ANKS6 | ZBTB8A | SLC14A2 | FNDC5 |
| RAB32 | PECR | RGL3 | XKR3 | RAMP2-AS1 |
| SIDT2 | GGTA1P | SEMA3G | DMD | RNU6-853P |
| OPN3 | FCGR2C | SLC22A16 | SLC25A48 | SRRM3 |
| NUDT16 | FAM212B | PHOSPHO1 | IL17RB | SEMG2 |
| CAMK2D | TGFA | NEK10 | TSPAN7 | SERPINB4 |
| SH3PXD2A | ADPGK-AS1 | PLCB1 | NKD1 | RCOR2 |
| SCIMP | ADAM12 | GPRC5C | CLEC4G | GPR143 |
| SLC26A6 | MTUS1 | PLEKHA6 | BIK | C19orf33 |
| TTYH2 | CD209 | CCL28 | DLG3 | STK32A |
| MAF | HOMER2 | UCP3 | SIGLEC6 | RAMP2 |
| CCNH | FAM110B | TMEM26 | LINC00885 | ANKRD20A1 |
| SOWAHC | RAMP1 | TTN | IGHE | WISP1 |
| BCL2L11 | SLC37A3 | SCD5 | FOXQ1 | LINC01122 |
| EPB41L2 | RPIA | ZNF365 | GLP2R | CDX1 |
| ALDH1A2 | DACT1 | SIGLEC12 | PRKCQ | MB |
| HN1 | HES6 | B3GALNT1 | GATA3 | TBX2 |
| PPFIBP1 | PTGFRN | SDPR | ANKRD55 | TMEM200A |
| TLE1 | PODXL | GCNT3 | SNORD125 | P2RY12 |
| KMO | NAPSB | PARM1 | HRH4 | GAL3ST1 |
| BPNT1 | CACNA1G | SLC30A4 | DNAH3 | B3GNT6 |
| PPM1L | BCL7A | CRB2 | DNAI1 | HHLA2 |
| MAOA | AKAP12 | PNPLA3 | PLXDC1 | TMC3 |
| SIGLEC14 | XXYLT1 | CDH1 | NPAS2 | IGHEP1 |
| CTNNAL1 | AKAP5 | LINC00475 | RBM11 | PLA2G5 |
| PCSK5 | TMTC4 | STAMBPL1 | SCUBE1 | GABRG2 |
| FRMD4A | GPR141 | SEBOX | DUOX1 | S1PR5 |
| PELP1 | CHN2 | VTN | PRKCQ-AS1 | DUXAP2 |
| NIPA1 | MANEAL | GALNT18 | LIMS2 | ABCC13 |
| PPARG | MS4A6E | ENO1-IT1 | DAAM2 | CRH |
| KCNK6 | MEX3B | ASTN2 | GPT | OR8G3P |
| ODF2 | S100A1 | NME8 | SULT1C2P1 | FGL1 |
| HOPX | LRP5 | LPO | TRIM71 | SPTSSB |
| BACE1 | FARP1 | CAPN14 | C9orf24 | PPP1R14A |
| DHRS11 | DIP2C | SOGA2 | TRPM1 | CIB4 |
| FAM126A | CD180 | MOCOS | CHRNA3 | KLRG2 |
| CARD9 | AIG1 | CABLES1 | S100B | SLC16A9 |
| SYNJ2 | GPR35 | RUFY4 | CCDC151 | KRT3 |
| PPFIBP2 | NEB | CD200R1 | FAM19A4 | HSD3B1 |
| RABEPK | CFP | MEST | NAALADL2 | CFHR1 |
| FGD2 | GPR146 | ZNF711 | SH3TC2 | FAM170B |
| SNAI3 | RAB33A | KCNK5 | IL1RL2 | SFRP1 |
| MAP1A | TAGLN | LEPREL2 | BAI2 | |
| ZNF366 | VWCE | ACSM5 | SLC7A2 | |
| GAS2L3 | RRS1 | STON2 | CNGA1 | |
| SLC45A4 | XPNPEP2 | F13A1 | TMEM236 | |

TABLE 9
Most expressed genes for M2c macrophages

| | | | | |
|---|---|---|---|---|
| MMP7 | LIN7A | CXADR | KIAA1211L | CHRNA6 |
| TIMP1 | GLDN | GXYLT2 | PCDHGA11 | BCYRN1 |
| CD163 | NAIP | WASF1 | PHEX | ZFPM2 |
| MARCO | MMP8 | NPDC1 | CRYAB | PRL |
| VCAN | CD226 | DNAH17 | AR | CHGA |
| SEMA6B | PTPRN | SPINK1 | PVALB | LRRC2 |
| SH3PXD2B | TSPAN13 | PARVA | NMNAT2 | DNAH17-AS1 |
| PLAU | PCOLCE2 | CLEC1A | SLC16A2 | OR13A1 |
| SLC25A19 | LIMCH1 | TDO2 | FAP | PRG3 |
| COL22A1 | PLOD2 | LAMC2 | C10orf55 | RNF175 |
| SLC12A8 | CD300E | CCR2 | BNIP3P1 | PROK2 |
| PROS1 | F5 | GRPR | DDAH1 | AWAT2 |
| FPR1 | CASC15 | CD163L1 | BICC1 | SNCB |
| PDPN | LGI2 | FGD1 | SPATA20P1 | KCNK15 |
| SRPX2 | SH2D4A | EDNRB | C7orf63 | |

Other suitable markers are described in Marc Beyer et al., “High-Resolution Transcriptome of Human Macrophages,” 7(9) PLOS ONE e45466 (2012) and Fernando O. Martinez et al., “Transcriptional Profiling of the Human Monocyte-to-Macrophage Differentiation and Polarization: New Molecules and Patterns of Gene Expression,” 177 J. Immunol. 7303-11 (2006).

Although steps S106, S108, and S110 were described in the context of cDNA synthesis and quantitative PCR, one of ordinary skill in the art will recognize that gene expression can be measured using other tools and techniques such as microarrays, RNA Sequencing (RNA-seq), and the like.

In step S112, a function of one or more of the expression levels of the measured markers is calculated. Various methods are described herein, including in the context of step S1802 in FIG. 18. In one embodiment, the function is a ratio. For example, the ratio can be a ratio of a single marker (e.g., IL1B) associated with M1 macrophage activity and a single marker (e.g., CD163 or CD206) associated with M2 macrophage activity. In other embodiments, the ratio is a ratio of a function (e.g., a weighted summation) of a plurality of markers associated with M1 macrophage activity to a function (e.g., a weighted summation) of a plurality of markers associated with M2 macrophage activity. The function can be a linear (i.e., first-order) function or can be a non-linear (e.g., second-order, third-order, fourth-order, parabolic, exponential, logarithmic, and the like) function. Although certain exemplary linear functions are described below, other linear functions such as a canonical correlation (in which linear coefficients such as αᵢ and βⱼ are optimized such that the correlation between markers of each phenotype are maximized) are within the scope of the invention.

For example, gene expression values for five M1 and five M2 markers can be combined into a single number using a linear sum of M1 markers divided by a linear sum of M2 markers, after multiplication of each expression value by a coefficient chosen to enhance or diminish the contribution of its corresponding gene according the following formula.

$$\frac{M 1}{M 2} score = \frac{{Σ}_{i = 1}^{5} {α}_{i} {G}_{i}}{{Σ}_{j = 1}^{5} {β}_{j} {G}_{j}}$$

Here, Gᵢ and Gⱼ are genes associated with M1 and M2 macrophages cultured in vitro, respectively, and αᵢ and βⱼ are coefficients obtained using the following methods summarized in Table 10.

TABLE 10
Summary of methods used for the conversion of gene expression data into a
combinatorial M1/M2 score.

| Name of method | Purpose | Approach |
|---|---|---|
| PCA | To capture maximum variance between | Principal Component Analysis |
| | M1 and M2 by magnifying differences of | |
| | the most important genes | |
| Weighted scaling | To give greater weight to those genes that | Using t-statistics |
| | are expressed at very different levels by | |
| | M1 and M2 | |
| Greedy | To maximize the difference between M1 | Non-linear Optimization |
| | and M2 as two distinct populations | |
| Mean-centering | To equalize contribution of all genes | Each gene was normalized to |
| | | its in vitro expression |
| Linear sum | To account for natural differences in the | All coefficients set to one |
| | level of expression by M1 and M2 | |
| IL1B/CD206 | To determine the major contributors and | Correlation Matrix |
| | to reduce the number of genes | |
| IL1B/CD163 | Utilizing newly discovered importance of | The expression of IL1B was |
| | M2c in wound healing. | normalized to the expression |
| | | of CD163 |

In the first method, αᵢ and βⱼ were obtained from principal component analysis (PCA) performed on gene expression data of M1 and M2 macrophages cultured in vitro (“PCA method”). PCA is a mathematical algorithm that is frequently used in gene expression studies for dimensionality reduction and data visualization as discussed in M. Ringner, “What is principal component analysis?” 26(3) Nature Biotechnology 303-04 (March 2008) and M. Parka et al., “Several biplot methods applied to gene expression data,” 138 J. Statistical Planning and Inference 500-15 (2008). In brief, PCA finds new directions in dataset, referred to as principal components (PCs), by capturing most of the variation in dataset. PCs are defined as linear combinations of the original variables. Therefore, the original variables and the transformed data can be visualized in a 2D or 3D vector space built upon the first two or three PCs, respectively.

In a “weighted scaling” method, αaᵢ and βⱼ are chosen to be t statistics obtained from a Student's t-test performed to compare expression of the corresponding gene between M1 and M2 macrophages cultured in vitro. A higher t-statistic indicates a greater degree of difference between M1 and M2 macrophages. Thus, the weighted scaling approach aims to give more weight to those genes with higher levels of significance. Use of t statistics has been reported previously in formulation of linear predictor scores from gene expression data in G. Wright et al., “A gene expression-based method to diagnose clinically distinct subgroups of diffuse large B cell lymphoma,” 100(17) P.N.A.S. 9991-96 (2003).

Alternatively, the “greedy method” seeks αᵢ and βⱼ such that the p value of a t-test performed on the combinatorial score of M1 and M2 macrophages cultured in vitro was minimized (“Greedy method”). The greedy method iteratively solves for coefficients such that the difference between M1 and M2 macrophages cultured in vitro was maximized; i.e., the p value of the t test on the combinatorial score of M1 and M2 macrophages cultured in vitro was minimized. For example, one could first set up a t-test comparing the scores, with coefficients of αᵢ and βⱼ, of in vitro-derived M1 and M2 macrophage populations, and with an output of the p-value. Any optimization method can then be employed (such as the Solver add-in in MICROSOFT® EXCEL®, available from Microsoft Corporation of Redmond, Wash.) to find αᵢ and βⱼ such that the p-value is as small as possible, or that the difference between the scores for the M1 and M2 populations is as large as possible. These optimized coefficients αᵢ and βⱼ could then be used in the calculation of the scores for wound data.

In the “mean-centering” method, the inverse of the mean in vitro expression of each gene is used as its coefficient in the M1/M2 score to equalize contribution of all genes. This approach seeks to account for inherent differences between expression values of different genes and to prevent those genes that are naturally expressed at higher levels from possible masking of the expression of the rest of the genes. This approach was used to scale the expression values for genes, which are expressed at very different levels, to the same level so that one highly expressing gene would not mask all the others, for example.

For example, CD206 and CCL18 are both M2 markers, meaning their expression is significantly higher in M2 macrophages comparing to M1 macrophages, yet their expression values differ several orders of magnitude. On average, CD206 is expressed 162.84 and 2.25 times relative to house keeping gene GAPDH in M2 and M1 macrophages, respectively. CCL18, however, is expressed 1.07 and 0.02 times relative to house keeping gene GAPDH in M2 and M1 macrophages, respectively. In addition, for example, CCR7 and IL1B are both M1 markers, meaning their expression is significantly higher in M1 macrophages comparing to M2 macrophages, yet their expression values differ several orders of magnitude. On average, CCR7 is expressed 0.33 and 0.02 times relative to housekeeping gene GAPDH in M1 and M2 macrophages, respectively. IL1B, however, is expressed 0.04 and 0.0004 times relative to housekeeping gene GAPDH in M1 and M2 macrophages, respectively.

Therefore, the following steps can be utilized to process a typical sample (from a wound or other tissue) with exemplary expression values of [CCR7, IL1B, CD206, CCL18]=[0.16, 0.026, 1.99, 0.05] under the mean-centering approach. First, expression values of M1 markers are normalized to the average expression value of those markers in in vitro polarized M1 macrophages, i.e. [0.16/0.33=0.48, 0.026/0.04=0.65]. Second, expression values of M2 markers are normalized to the average expression value of those markers in in vitro polarized M2 macrophages, i.e. [1.99/162.84=0.001, 0.05/1.07=0.046]. Third, the M1/M2 score of the mean-centered values is calculated, i.e. M1/M2=(0.48+0.65)/(0.001+0.046)=24.04.

In a “linear sum” method, all of the coefficients were set to 1.

Steps S102-S112 can be repeated again after a period of time in order to assess the change in the ratio of M1 to M2 macrophage activity over time.

In step S114, the outputs of the functions (e.g., ratios) can be compared. The comparison can be a simple, absolute comparison of calculated ratios, a calculation of the linear rate of change, or can utilize a fold change to measure a ratio of the second ratio to the first ratio. Generally speaking, if the ratios remain substantially steady over the period of time, a transition from M1 to M2 macrophage activity has not occurred and the wound is not healing. If the ratio decreases (i.e., the M2 weighted sum increases relative to the M1 weighted sum or the M1 weighted sum decreases relative to the M2 weighted sum), the transition from M1 to M2 macrophage activity is occurring and the wound will likely heal. Although the degree of change associated with healing and nonhealing wounds will vary between the functions applied to generate the M1 and M2 scores, healing wounds and nonhealing wounds scored using the six functions listed in Table 6 exhibited a MEAN+/−SEM fold changes of 0.29+/−0.07 and 4.09+/−0.83, respectively. Without being bound by theory, it is believed that, regardless of the method used to generate the M1 and M2 scores, the fold change of the M1:M2 ratio over time will be between 0 and 1 for healing wounds and greater than 1 (e.g., between 1 and 20, between 1 and 25, between 1 and 30, and the like) for nonhealing wounds.

The diagnostic threshold for a particular function can be computed using tools and techniques such as receiver operating characteristic (ROC) curves.

Implementation in Computer-Readable Media and/or Hardware

The methods described herein can be readily implemented in software that can be stored in computer-readable media for execution by a computer processor. For example, the computer-readable media can be volatile memory (e.g., random access memory and the like) and/or non-volatile memory (e.g., read-only memory, hard disks, floppy disks, magnetic tape, optical discs, paper tape, punch cards, and the like).

Additionally or alternatively, the methods described herein can be implemented in computer hardware such as an application-specific integrated circuit (ASIC).

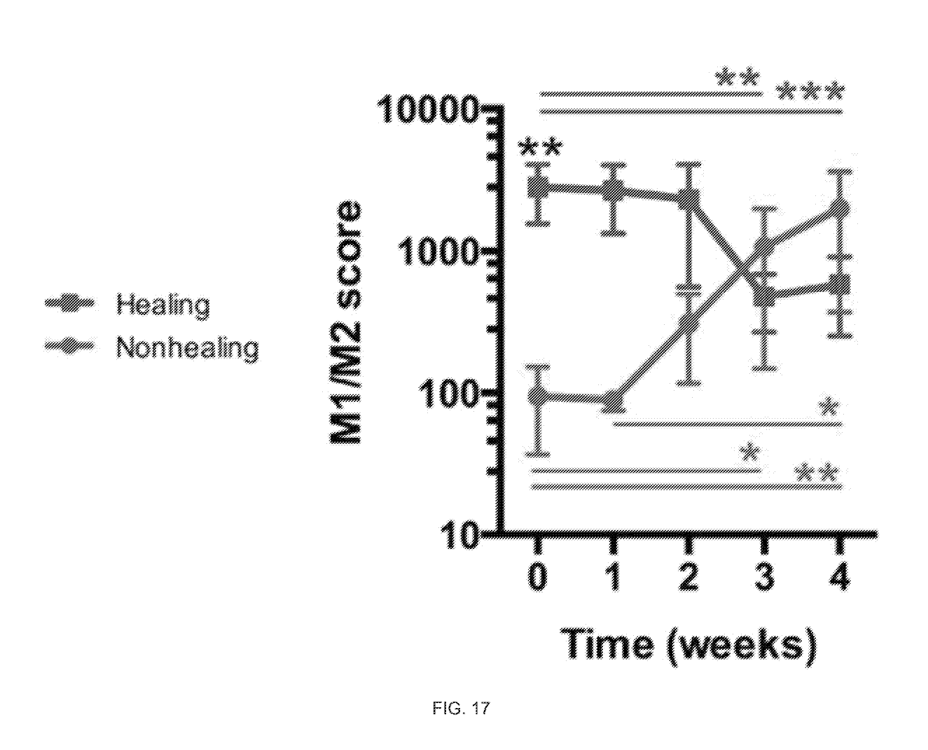

Referring now to FIG. 13, another embodiment of the invention provides a system 1300 for assessing a wound. System 1300 can include a computing device 1302 (e.g., a general-purpose computer, a tablet, a smartphone, and the like). Computing device 1302 can be programmed with software as discussed herein to implement the methods described herein. System 1300 can also include a thermocycling device for performing quantitative PCR. Suitable thermocyclers are available from Life Technologies of Grand Island, N.Y. Computing device 1302 can be in communication with thermocycler 1304 via wired or wireless communication.

### High-Throughput Screening for Identification of M1, M2a, and M2c Macrophages

Another aspect of the invention provides a high-throughput (HTP) screening assay and system for analyzing healing and wound healing properties, such as identifying macrophage phenotype, predicting healing progression, analyzing response to a stimulus, etc. The HTP assay allows screening of expression transcripts, proteins, protein activity, functional response to a stimulus, etc. of multiple samples.

The HTP screening assay refers to the analysis of at least two samples simultaneously, iteratively, concurrently, or consecutively. In one embodiment, the number of samples assayed simultaneously is in the range of 1-10,000 samples. In another embodiment, the following ranges of sample number are assayed in the HTP screen: 1-5,000, 1-2,500, 1-1,250, 1-1,000, 1-500, 1-250, 1-100, 1-50, 1-25, 1-10, 1-5, 7,500-10,000, 5,000-10,000, 4,000-10,000, 3,000-10,000, 2,000-10,000, 1,000-10,000, 500-10,000, 100-1,000, 200-1,000, 300-1,000, 400-1,000, 500-1,000, and any other number of samples therebetween.

The HTP system can include, but is not limited to, measurement devices, robotic pippettors, robotic samplers, robotic shakers, data processors and storage devices, data processing and control software, liquid handling devices, incubators, detectors, hand-held detectors, and the like. For the purposes of automation, the number of samples tested at one time can correspond to the number of wells in a standard plate (e.g., 6-well plate, 12-well plate, 96-well plate, 384-well plate, and the like). The samples can be obtained from a plurality of cells, tissues, individuals, or from a plurality of samples obtained from a single individual.

In one embodiment, the HTP screening assay permits the analysis and/or prediction of healing or wound healing properties. In another embodiment, the HTP screening assay permits the identification of macrophage phenotype, such as M0, M1, M2a, M2b, M2c macrophage. In yet another embodiment, the HTP screening assay allows for the analysis and/or identification of response to a stimulus, such as a titration of a therapeutic, sensitivity or response to a library of therapeutics, or other agents. In still another embodiment, the HTP screening assay allows for comparison of gene expression signatures.

In one embodiment, the method includes obtaining one or more measurements as described elsewhere herein and comparing the measurements to analyze and/or predict healing or wound healing properties in the wound. The measurements can be obtained from one or more macrophage phenotype populations. In another embodiment, the method includes obtaining one or more measurements from a wound, a non-wound, different wounds, a healing wound, a non-healing wound, and any combination thereof. In yet another embodiment, multiple measurements are taken from the same sample for comparison. The measurements can be taken in a time course over a defined period of time, seconds, minutes, hours, days, weeks, etc.

In another embodiment, the method includes obtaining one or more samples and/or preparing the samples for analysis. The HTP screening assay as described herein can utilize techniques previously used in the art to obtain and prepare the samples for analysis. The preparation of the samples can depend on the measurement(s) to be obtained, the type of sample, and any other property dependent on the HTP screen.

In another embodiment, the method includes analyzing the phenotype of macrophages cultivated in vitro.

In another embodiment, the method includes comparing the measurements as described elsewhere herein. The HTP screening assay allows for the comparison and output analysis of multiple measurements of the same property, multiple properties, or a combination thereof.

In one aspect, the HTP screening includes a method of analyzing and/or predicting healing or wound healing properties. In one embodiment, the method includes obtaining one or more measurements of one or more macrophage phenotype populations in a wound and comparing the measurements to analyze and/or predict a healing or wound healing property.

In another aspect, the HTP screening includes a method of identifying macrophage phenotype in a wound. In one embodiment, the method includes obtaining one or more measurements of one or more macrophage phenotype populations and comparing the measurements to identify macrophage phenotype. In one embodiment, comparing the measurements identifies a primary or predominant macrophage phenotype in the wound, such as M0, M1, M2a, M2b, M2c macrophage.

In still another aspect, the HTP screening includes a method of differentiating a macrophage phenotype from another macrophage phenotype. In one embodiment, the method includes obtaining one or more measurements and comparing the measurements to differentiate M0, M1, M2a, M2b, or M2c macrophages from the other phenotypes. In this embodiment, an expression profile/signature and/or protein levels are measured and compared to differentiate the macrophage phenotypes. For example, an expression profile/signature includes expression or protein levels of one or more of CD163, MMP7, MMP8, MMP9, MMP12, TIMP1, VCAN, PLAU, PROS1, SRPX2, NAIP, and F5 to differentiate M2c macrophage from one or more other phenotypes. In another example, an expression profile/signature includes expression of SOD2 to differentiate M1 from one or more other phenotypes or expression of CCL22 to differentiate M2a from one or more other phenotypes. Analysis of the expression profile/signature and/or protein levels can also predict a healing or wound healing property, response of macrophage to a stimulus, or other property described herein.

In yet another aspect, the HTP screening includes a method of analyzing and/or identifying a response of macrophage in a wound or from macrophages cultivated in vitro to a stimulus. In one embodiment, the method includes screening a library of therapeutics or small molecules by analyzing a response of the macrophage exposed to a stimulus, such as therapeutic or small molecule. One or more of the samples can be exposed to stimulus before, during or after measurement. Additional measurements may be obtained on the same samples any time after exposure.

### Methods of Predicting Tumor Progression

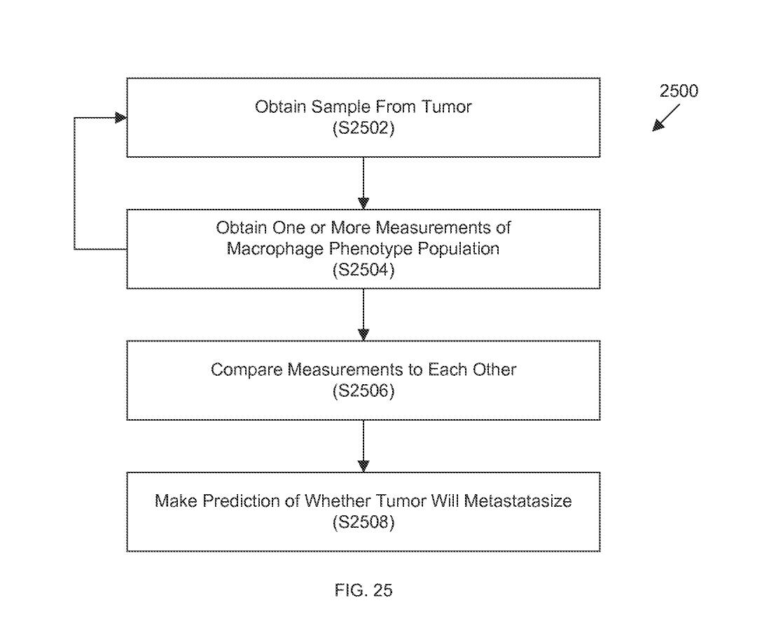

Referring now to FIG. 25, another aspect of the invention provides a method 2500 of predicting tumor progression. The current standard of care for many cancer patients involves removal of tumors followed by aggressive treatment as prophylaxis against undetected metastases. These aggressive treatments have significant side effects on the patient.

In step S2502, a sample is obtained from the tumor. This sample can be obtained before, during, or after removal of the tumor using various biopsy, surgical, and/or laboratory tools and techniques.

In step S2504, one or more measurements of macrophage phenotype population are obtained, e.g., using the methods described herein. In one embodiment, measurements of the M1 and M2 macrophage populations (e.g., M1 and M2a, M1 and M2c, and the like) are obtained.

In step S2506, the measurements are compared to each other. In one embodiment, this comparison is expressed as a ratio as discussed herein.

In step S2508, a prediction of whether the tumor will metastasize is made based on a result of the comparing step S2506. Without being bound by theory, it is believed that an M1:M2, M1:M2a, or M1:M2c ratio exceeding a threshold that can be determined through analysis of data obtained using a particular panel of biomarkers can be indicative of a tumor that has a low likelihood of metastasis. This prediction can be used to inform clinical decisions regarding what prophylactic measures should be undertaken (if any).

### WORKING EXAMPLES

### Materials and Methods

### Experimental Design

A panel of genes were selected that were highly indicative of macrophage phenotype using macrophages cultivated and polarized in vitro towards the M1 and M2 phenotypes. Next, a number of algorithms for converting expression data of 10 different genes into a combinatorial score were evaluated. These algorithms were applied to debrided wound tissue obtained from human diabetic foot ulcers over the course of 30 days from the initial visit in order to describe differences in macrophage behavior between healing and nonhealing diabetic wounds and in comparison to healing acute wounds. A publicly available dataset from a longitudinal study of wound healing in acute burn wounds in humans provided in Greco was used as the healing acute wound data.

### Preparation and Characterization of Polarized Macrophages in Vitro

Freshly isolated primary monocytes, purified via negative selection from human peripheral blood mononuclear cells, were purchased from University of Pennsylvania Immunology Core. Monocytes were cultured and polarized in vitro into M1 or M2 macrophages as previously described in Spiller 2014. In brief, monocytes were cultured with monocyte colony stimulating factor (MCSF; 20 ng/ml) for 5 days to differentiate them into macrophages. Then, M1 or M2 polarization was achieved by addition of interferon-gamma (IFNγ; 100 ng/ml) and lipopolysaccharide (LPS; 100 ng/ml) for M1 or Interleukin-4 (IL-4; 40 ng/ml) and Interleukin-13 (IL13; 20 ng/ml) for M2. After 2 days of polarization, RNA was extracted for gene expression analysis of M1 and M2 markers by real time quantitative reverse transcription polymerase chain reaction (qRT-PCR) as in Spiller 2014.

### Patient Enrollment

Thirteen patients with chronic diabetic foot ulcers were recruited from the Drexel University Wound Healing Center in compliance with the study protocol reviewed and approved by the Drexel University Institutional Review Board. Participants were between 50-70 years of age and had at least one open wound on either a foot or lower extremity that had not healed for 8 weeks at the time of enrollment. Patients were excluded if they presented with signs and symptoms of a major infection, abscess, or untreated osteomyelitis. During the study, participants underwent standard wound care procedures determined by the physician, including weekly or biweekly wound debridement, standard length-times-width ruler measurement of wound size, and prescribed topical dressings. Participants were divided into two groups, healing and nonhealing, based on whether their wound was completely healed within 70 day from the initial visit. Only patients who returned for follow-up visits were included in this study in order to facilitate a longitudinal analysis of wound healing. Of these seven patients, three had wounds that completely closed over the course of 70 days and thus were designated “healing,” and four had wounds that did not heal and thus were designated “nonhealing.” Wound Sample Collection

Participants underwent wound debridement as part of standard wound care regimen during each visit to the clinic. Debrided tissue was immediately collected in RNALATER® solution to stabilize and protect the RNA content of the tissue. Samples were stored in RNALATER® solution at 4° C. overnight as per the manufacturer's suggestion, and were subsequently moved to −80° C. until further analysis by qRT-PCR.

RNA Extraction, Complementary DNA Synthesis, and qRT-PCR

Wound samples were thawed at room temperature and processed for RNA extraction using TRIZOL® Plus RNA purification kit according to the manufacturer's instructions. Extracted RNA was eluted in 30 μL of RNAse-free water and stored at −80° C. until synthesis of complementary DNA (cDNA) using the APPLIED BIOSYSTEMS® High-Capacity cDNA Reverse Transcription Kit available from Life Technologies. Lastly, quantitative analysis of expression of multiple markers of macrophage phenotype was performed using qRT-PCR with GAPDH as a reference gene, as previously described in Spiller 2014.

### Identification of M2c Macrophage Biomarkers

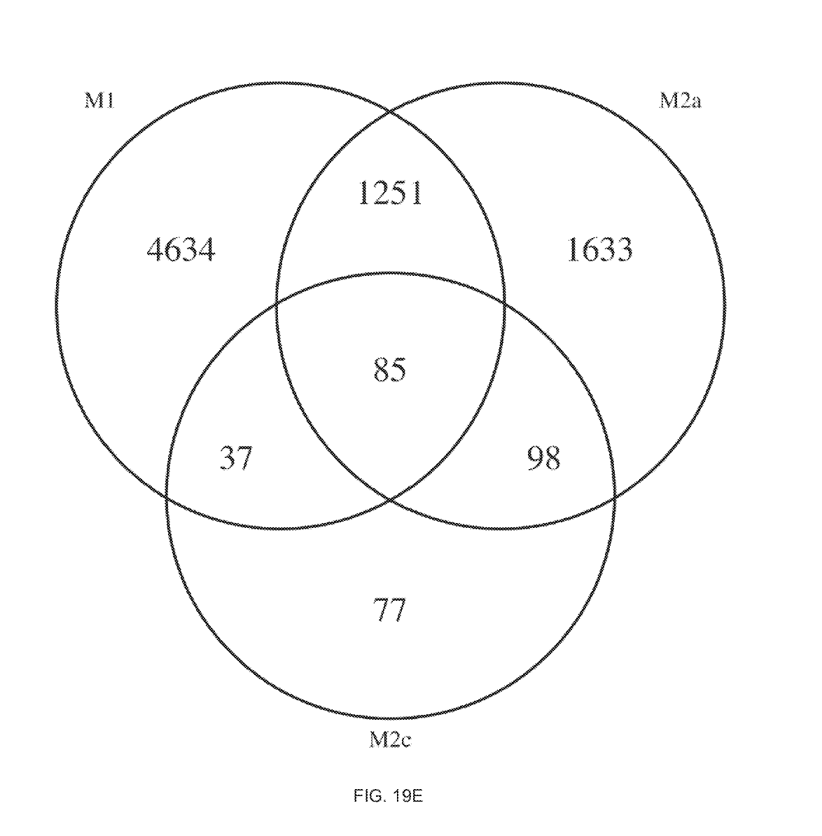

Referring now to FIGS. 19A-19C, RNA Sequencing (also known as RNA-Seq, Whole Transcriptome Shotgun Sequencing, or WTSS) was utilized to identify genes that are up- and down-regulated in M2c macrophages relative to M0 macrophages (comparison depicted in FIG. 19A), M1 macrophages (comparison depicted in FIG. 19B), and M2 macrophages (comparison depicted in FIG. 19C). Referring now to FIGS. 19D and 19E, Venn diagrams depict overlapping and distinct genes that are up-regulated and down-regulated, respectively, in M1, M2a, and M2c macrophages relative to MO macrophages.

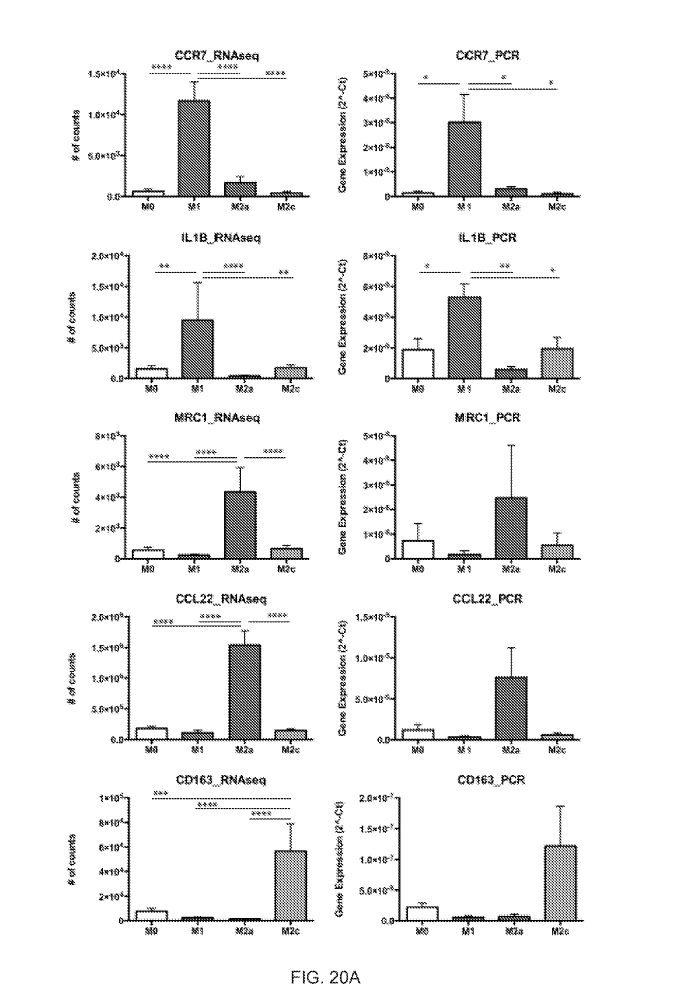

Referring now to FIGS. 20A and 20B, the genes identified through RNA-Seq were validated using RT-PCR. FIG. 20A depicts transcriptional profiles across M0, M1, M2a, and M2c macrophages for biomarkers of M1 macrophages (i.e., CCR7 and IL1B), M2a macrophages (MRC1 and CCL22), and M2c macrophages (CD163). FIG. 20B depicts transcriptional profiles across M0, M1, M2a, and M2c macrophages for biomarkers for newly discovered genes associated with M2c macrophages: TIMP1, Marco, VCAN, SH3PXD2B, and MMP8.

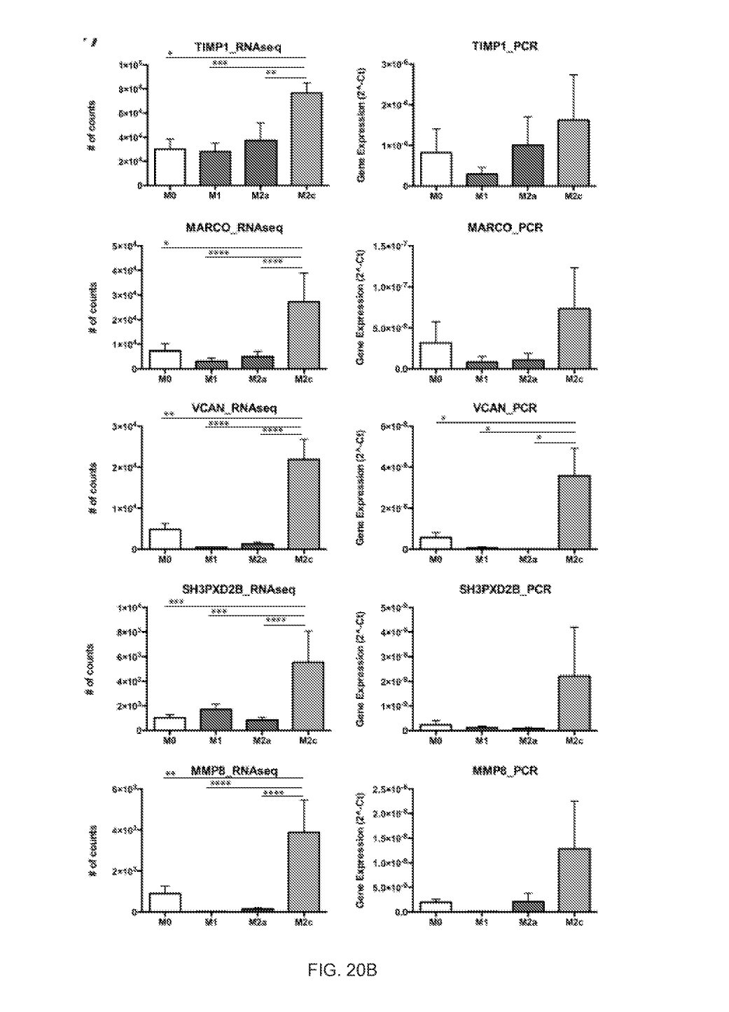

Referring now to FIG. 21, bar graphs of protein secretion (as determined by ELISA analysis of cell culture supernatant) for newly discovered M2c markers TIMP1, MMP7, and MMP8 are depicted.

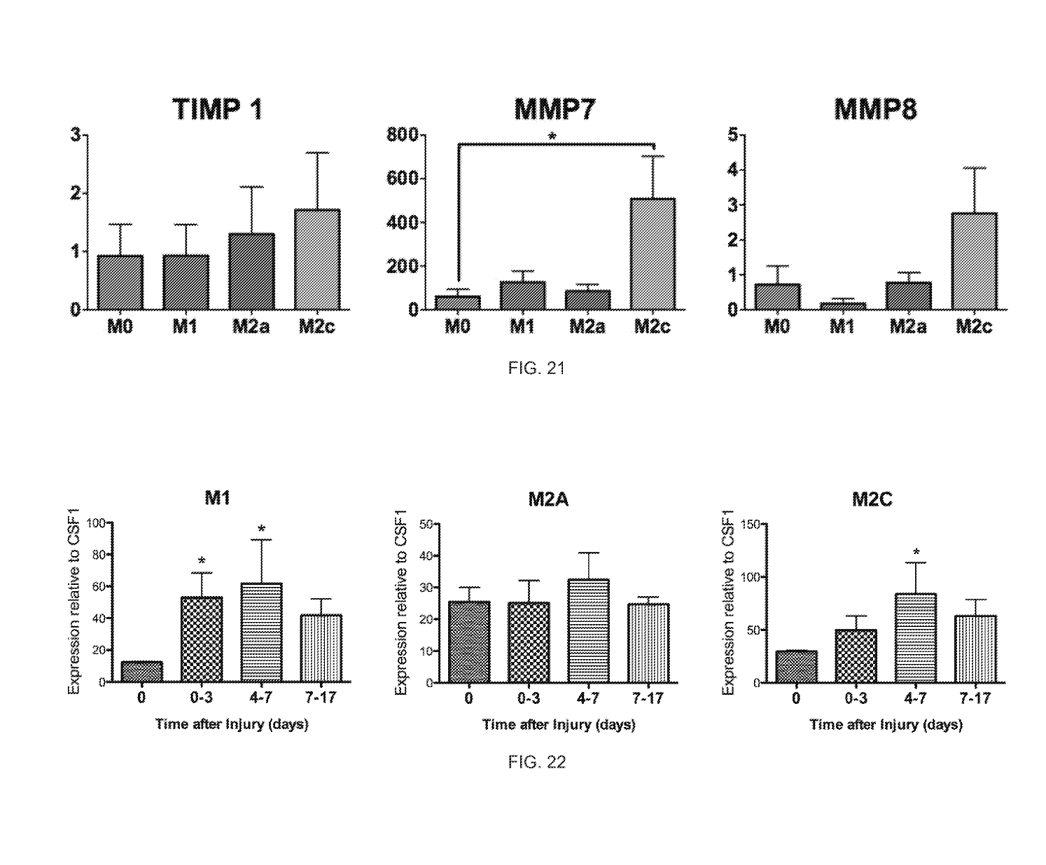

Referring now to FIG. 22, bar graphs of summed expression of raw data of ˜5 highly expressed genes of the M1, M2a, and M2c phenotypes, in publicly available data available from Greco 2010, showing that these signatures (as opposed to a ratio) can be used to track macrophage phenotype. Thus, these signatures can be used in a diagnostic assay to track healing without using a ratio. For example, increasing M2c values over time suggest a healing wound. In one embodiment, measurements obtained from a single sample or from multiple samples over time can be compared to a plurality of profiles to identify which pattern best fits the data (e.g., by minimizing sums of the squared deviations between the actual data and the average data in each model).

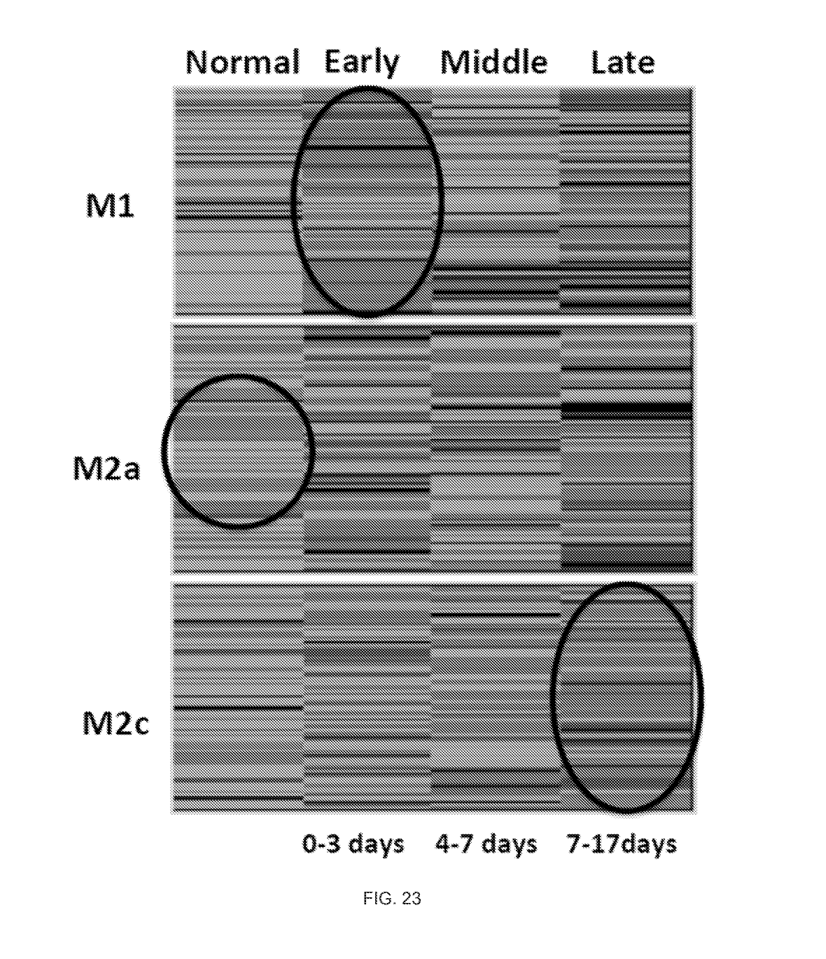

Referring now to FIG. 23, heat maps of the top 60 most highly expressed genes by the M1, M2a, and M2c phenotypes show that M1 markers are upregulated at early times after injury while M2a and especially M2c markers are upregulated at later times after injury, using publicly available data from Greco 2010. Thus, these signatures can be used to track macrophage phenotype and healing of a wound.

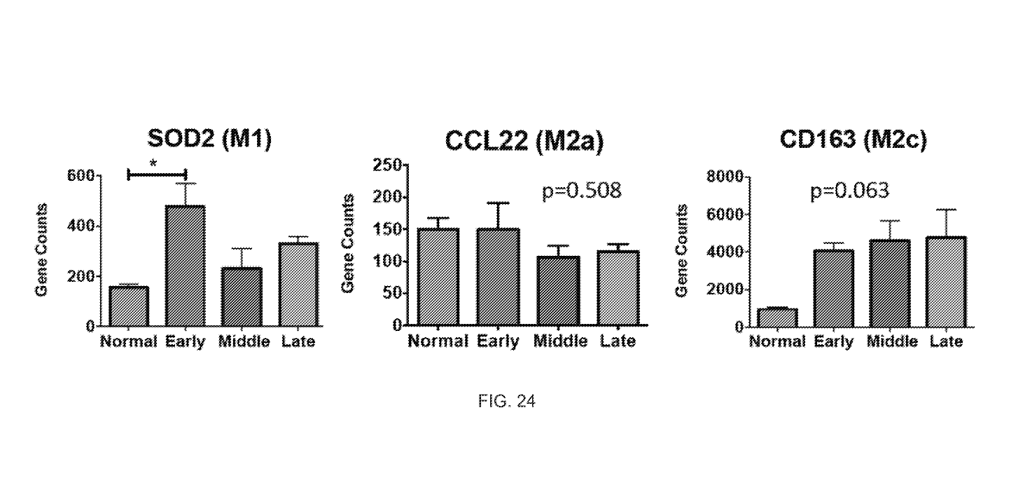

Referring now to FIG. 24, the most highly expressed M1, M2a, and M2c markers (SOD2, CCL22, and CD163, respectively) confirm that M1 macrophages are important at early times after wounding while M2c markers are important in both early and later stages. Thus, the ratio of M1 to M2 (both M2a and M2c) macrophages can be useful in predicting healing or nonhealing of a wound.

### Algorithms

Using the algorithms described herein, a score was calculated for each sample and plotted over time as fold change over the initial visit. The mean fold change for healing vs. nonhealing wounds was compared at 4 weeks after the initial visit, which is the amount of time recommended for assessment of the effectiveness of therapy and likelihood of healing by the guidelines provided by the Wound Healing Society for the treatment of diabetic ulcers, was assessed.

Conversion of a Panel of Macrophage Markers into a Single Score

Macrophages are complex and can exist as hybrid phenotypes exhibiting properties of both M1 and M2 macrophages and even other subtypes. Thus, a large number of genes may be required to accurately depict changes in their behavior. Applicant selected 9 genes and compared their expression levels in M1 and M2 macrophages cultivated in vitro as depicted in FIG. 1B.

Applicant next explored methods to convert the panel of 9 genes into a single score indicative of the relative M1-M2 character of the macrophages. To accomplish this, Applicant defined, for example, an “M1 over M2 score” as the linear sum of the expression of M1 genes divided by the linear sum of expression of M2 genes, resulting in higher scores for the M1 macrophages and lower scores for the M2 macrophages as depicted in FIG. 1C.

### Macrophage Gene Expression Profile in Human Healing and Nonhealing Diabetic Wounds

In order to investigate the accuracy of the M1 over M2 score in describing macrophage behavior over time in wounds, Applicant used the Greco burn data set, representing acute or “normal” healing wounds. Conversion of the raw data into the M1 over M2 score allowed for a single number that reflects the macrophage character of the tissue, while simultaneously normalizing the gene expression in such a way that the number would not be sensitive to wound heterogeneity. As depicted in FIG. 1D, the M1 over M2 score increases immediately after injury, and decreases back to baseline levels after 7 days of healing. Next, the linearly-summed M1-M2 score was used to track the M1-vs.-M2 characteristic of human diabetic ulcers by collecting the tissue obtained from wound debridement, a normal part of the standard wound care regimen, which would have otherwise been discarded. These patients had wounds that had not healed for at least 8 weeks at the time of enrollment. Samples were collected at each visit for at least 4 weeks or until the wound healed completely. After tracking the M1 over M2 score over time (represented as fold change from the initial visit), Applicant found that all wounds that healed over the course of the study exhibited a decreasing score over time as depicted in FIG. 1E, similar to healing acute wounds. In stark contrast, all wounds that failed to heal showed increasing M1 over M2 scores over time, corroborating reports that suggested an elevated inflammatory character in nonhealing chronic wounds and confirming animal models that suggested a defective M1-to-M2 transition in diabetic wounds. In fact, the mean fold change at 4 weeks after the initial visit was more than 60 times higher for nonhealing wounds compared to healing wounds as depicted in FIG. 1F. Without this score, the number of genes analyzed makes the data extremely difficult to interpret as seen in FIGS. 1G and 1H, which depict individual marker levels over time for typical healing and nonhealing wounds.

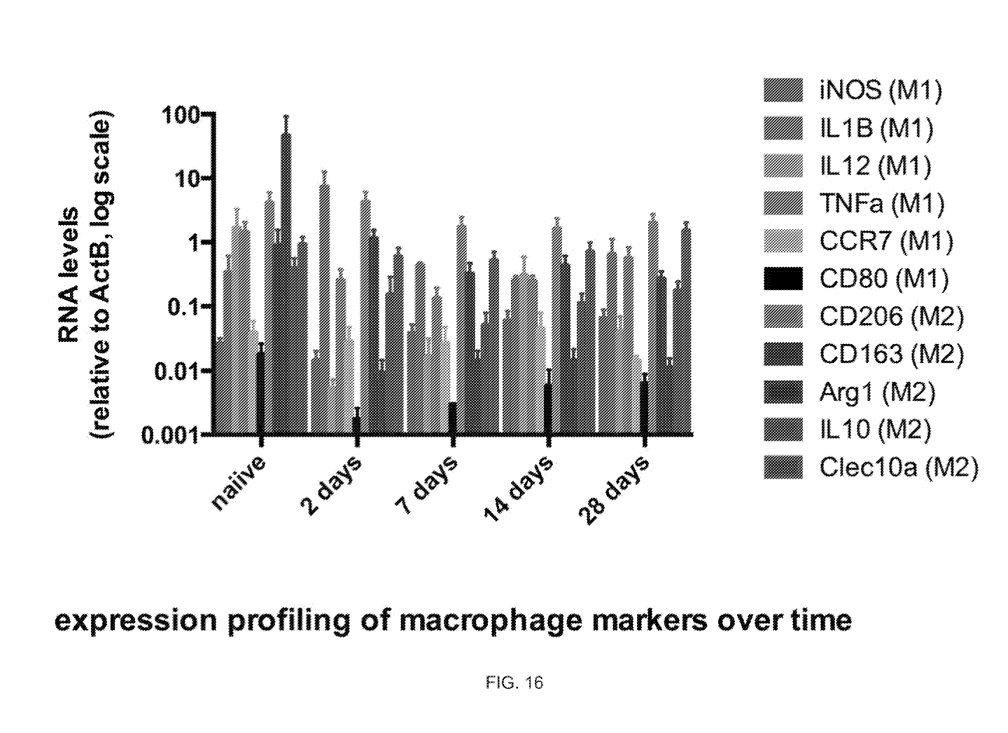

Interestingly, when a linearly-summed M1-M2 score was calculated using only those genes that were significantly different between M1 and M2 populations in vitro based on the data depicted in FIG. 1B, the M1-M2 score did not change significantly for healing wounds over time and the difference between healing and nonhealing wounds at 4 weeks was smaller than when all 9 genes were included. An M1 over M2 score calculated with only the most highly negatively correlated genes for M1 and M2 populations (CCR7 and TIMP3, R=−0.78 in FIG. 12) also did not yield differences between healing and nonhealing.

### Profile Analysis and the Confusion Matrix

To assess the potential utility of the proposed algorithms in a diagnostic assay and to compare their relative performance, profile analysis was performed by fitting a linear curve through the data points to obtain the score for each patient as a function of time. The average fold changes were then compared between healing and nonhealing wounds over time for as long as 4 weeks after the initial visit. To test the hypothesis that healing wounds would show a decrease in the relative proportion of M1/M2 macrophages and to assess the predictive functionality of each method over time, the threshold of the fold change was set to 1. To explore the possibility of predicting healing outcomes earlier than 4 weeks, which is the current clinical standard based on wound size, the true positive rate, true negative rate, positive predictive value, negative predictive value, and accuracy were calculated over time based on the confusion matrix for each method, and the true positive rate was plotted versus the false positive rate (defined as one minus true negative rate) over 1-4 weeks.

### Statistical Analysis

MATLAB® software (available from The MathWorks, Inc. of Natick, Mass.) was used for PCA and curve fitting. The Greedy method was executed in MICROSOFT® EXCEL® using the GRG nonlinear solver. A correlation matrix was plotted using the corrplot package in R software. All other statistical analyses were performed in GRAPHPAD™ PRISM™ 6 (available from GraphPad Software, Inc. of La Jolla, Calif.). Data are shown as mean±SEM and p<0.05 was considered significant. Student's t-test was used to compare M1 and M2 populations in vitro, as well as healing and nonhealing wounds at each time point. Grubb's test was used to identify the outlier in M2 macrophages polarized in vitro, as indicated.

### Results

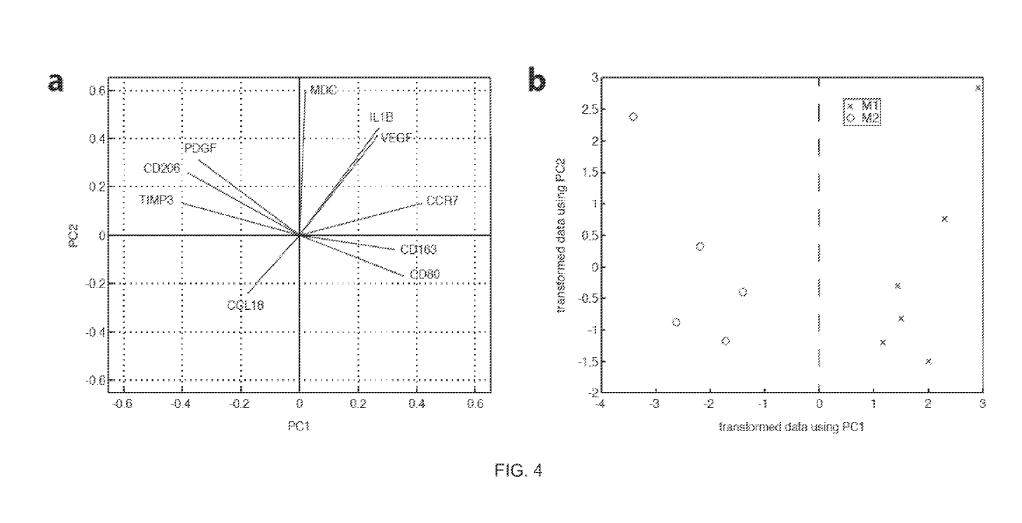

According to the guidelines provided by the Wound Healing Society, a 40% reduction in wound size after 4 weeks is suggested as a predictor of healing in patients with diabetic ulcers. In order to compare changes in wound size between healing and nonhealing chronic diabetic ulcers, fold changes of wound size relative to the initial visit for 30 days after the first visit were compared as depicted in FIG. 2. In agreement with previous findings, change in wound size appeared not to be a reliable predictor of healing outcomes as the mean fold change over day zero were not significantly different between the two groups at 4 weeks (p=0.58).

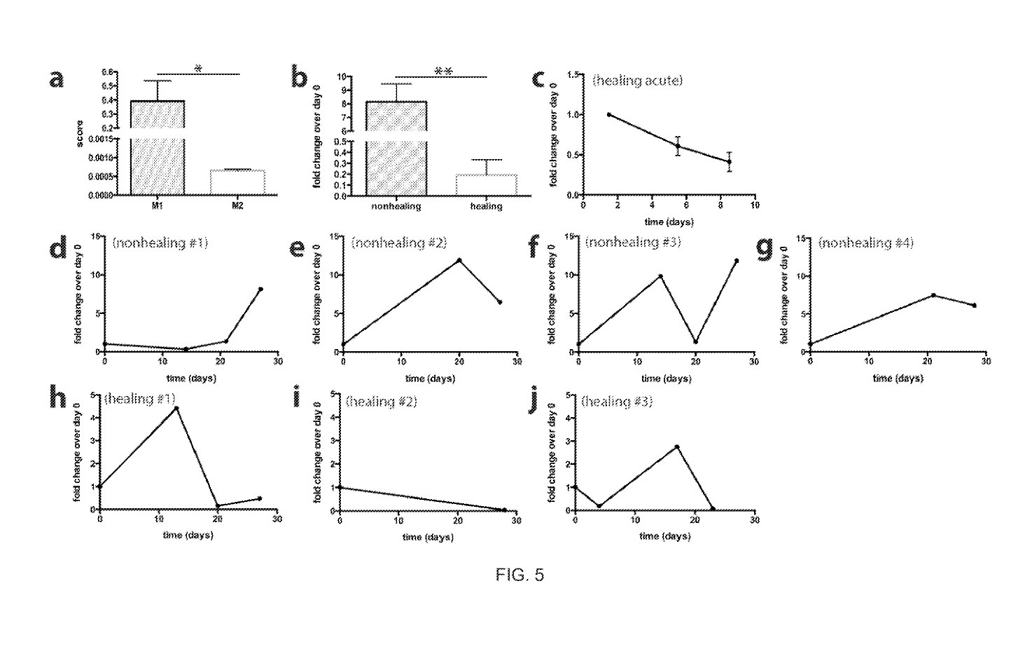

Ten genes were selected and their expression levels compared between the two phenotypes as depicted in FIG. 3. VEGF, CCR7, CD80, and IL1B were selected as M1 markers, and CCL18, CD206, MDC, PDGF, TIMP3, and CD163 were selected as M2 markers. Box and whisker plots of fold change expression over GAPDH revealed higher expression of all M1 markers in M1 macrophages compared to M2 macrophages, although only CCR7 (p<0.0001) and CD80 (p<0.001) were significant. Similarly, all M2 markers, with the exception of CD163, were expressed higher in M2 macrophages compared to M1 macrophages with only CD206 (p<0.05), PDGF (p<0.05), and TIMP3 (p<0.01) being significant. Interestingly, CD163 was expressed at significantly higher levels by M1 macrophages (p<0.01), even though it has been previously shown to be a robust marker of a subset of M2 macrophages, those polarized by IL10 and referred to as M2c in Spiller 2014. Because differentiation between the M2 subtypes was not intended in this study, CD163 was considered an M1 marker in the remainder of this Working Example.

P-values of the difference between healing and nonhealing wounds at 4 weeks for ratios of single markers of M1 macrophage activity to single markers of M2 macrophage activity are presented in Table 11.

TABLE 11
P-value of the Difference Between Healing and Nonhealing at
4 Weeks for Ratios of Single Markers

| | | P-value of the Difference Between Healing |
|---|---|---|
| M1 Gene | M2 Gene | and Nonhealing at 4 Weeks |
| VEGF | CCL18 | 0.368625445 |
| VEGF | CD206 | 0.327864313 |
| VEGF | MDC | 0.381608604 |
| VEGF | PDGF | 0.216295875 |
| VEGF | TIMP3 | 0.78106261 |
| VEGF | CD163 | 0.353863894 |
| CCR7 | CCL18 | 0.370353945 |
| CCR7 | CD206 | 0.371951557 |
| CCR7 | MDC | 0.298055089 |
| CCR7 | PDGF | 0.32543361 |
| CCR7 | TIMP3 | 0.744078158 |
| CCR7 | CD163 | 0.85056864 |
| CD80 | CCL18 | 0.456329225 |
| CD80 | CD206 | 0.932151157 |
| CD80 | MDC | 0.163398836 |
| CD80 | PDGF | 0.657799104 |
| CD80 | TIMP3 | 0.304039946 |
| IL1B | CD163 | 0.40178575 |
| IL1B | CCL18 | 0.252829412 |
| IL1B | CD206 | 0.046251377 |
| IL1B | MDC | 0.13204204 |
| IL1B | PDGF | 0.139303456 |
| IL1B | TIMP3 | 0.081109122 |
| IL1B | CD163 | 0.011682275 |

P-values of the difference between healing and nonhealing wounds at 4 weeks for ratios of linear summations of one or more markers of M1 macrophage activity to linear summations of a plurality of markers of M2 macrophage activity are presented in Table 12.

TABLE 12
P-value of the Difference Between Healing and Nonhealing at
4 Weeks for Ratios of Single Markers

| | | P-value of the Difference |
|---|---|---|
| | | Between Healing and |
| M1 Gene(s) | M2 Genes | Nonhealing at 4 Weeks |
| IL1B | TIMP3 + CD163 | 0.009924383 |
| VEGF + CCR7 + CD80 | CD206 + TIMP3 | 0.047132339 |

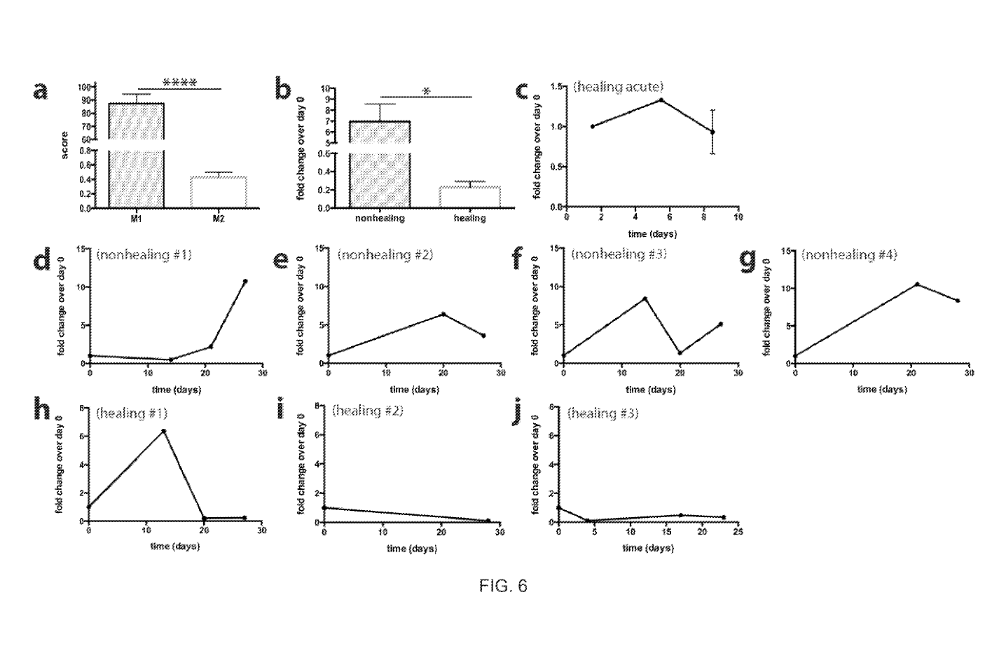

In order to further explore the in vitro data and to visualize similarities and differences between M1 and M2 macrophages, principal component analysis (PCA) was performed as depicted in FIG. 4. The first two PCs collectively captured 73% of the total variance in our dataset. The coordinates of gene vectors on the PCA biplot depicted in Panel (a) of FIG. 4 represent the coefficients for the first two PCs. Vectors that lie in similar direction on the biplot have high positive correlation. For example, it is evident from the biplot that CD163 is highly positively correlated with the M1 markers CCR7 and CD80. Moreover, the biplot demonstrates that M1 and M2 markers, except for MDC, are positioned in opposite directions with respect to first principal component (PC1), suggesting that PC1 has the potential to be used for classification of M1 and M2 macrophages. MDC is almost parallel to second principal component (PC2), which is by definition uncorrelated with PC1. Therefore, in agreement with what was observed in box and whisker plots of FIG. 3, it appears that among the 10 selected genes, MDC is the least effective marker for differentiating between M1 and M2 macrophages. The PCA sample plot, on the other hand, demonstrates samples with similar gene profiles as nearly located points and, therefore, can be used to examine the relationship between samples. As depicted in Panel (b) of FIG. 4, in this case, PC1 was capable of successfully classifying samples into M1 and M2.

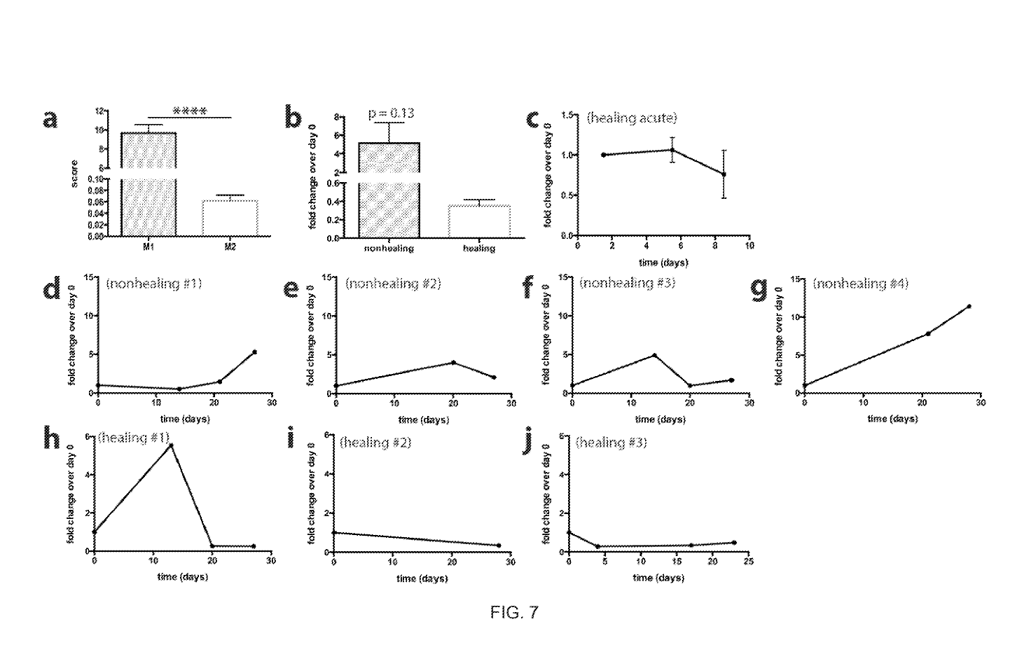

With gene expression profile of in vitro polarized M1 and M2 macrophages serving as signature profiles of the two extremes on the spectrum of phenotypes along which macrophages exist, a combinatorial score was defined based on gene expression data of 5 M1 and 5 M2 macrophage genes. The M1/M2 score was then applied to in vitro data of polarized macrophages, healing and nonhealing chronic diabetic ulcers over the course of 4 weeks, and public data from acute healing wounds. In all methods (depicted over FIGS. 5-10), the M1/M2 score was found to be significantly higher for M1 macrophages compared to M2 macrophages cultured in vitro, except for IL1B/CD206. Interestingly and yet for all six methods, the score appeared to increase over time for nonhealing chronic diabetic ulcers, and to decrease for healing ones with some fluctuations in between. Comparison of fold change over day zero between healing and nonhealing wounds revealed a significant difference at 4 weeks, except for greedy and mean-centering methods. Furthermore, and in support of the hypotheses, decrease of M1/M2 score over time in healing chronic diabetic ulcers resembled the trend observed in acute healing wounds.

In order to develop a score that generates a difference between M1 and M2 macrophages by weighing each gene according to its share of the total variation, PCA was used to obtain the linear combinations of M1 and M2 genes. The absolute value of the PC1 coefficients indicates contribution of each gene in capturing most of variance, as well as its ability to classify samples into M1 and M2. The sign of each coefficient, however, is not of interest unless visualization on the PC vector space is intended. Therefore, the absolute values of the PC1 coefficients were used to define the M1/M2 score as depicted in FIG. 5. As depicted by box and whisker plots as well as PCA, the difference between expression levels of M1 and M2 macrophages is more significant for some genes (such as CCR7, CD80, CD163, and TIMP3) than other genes.

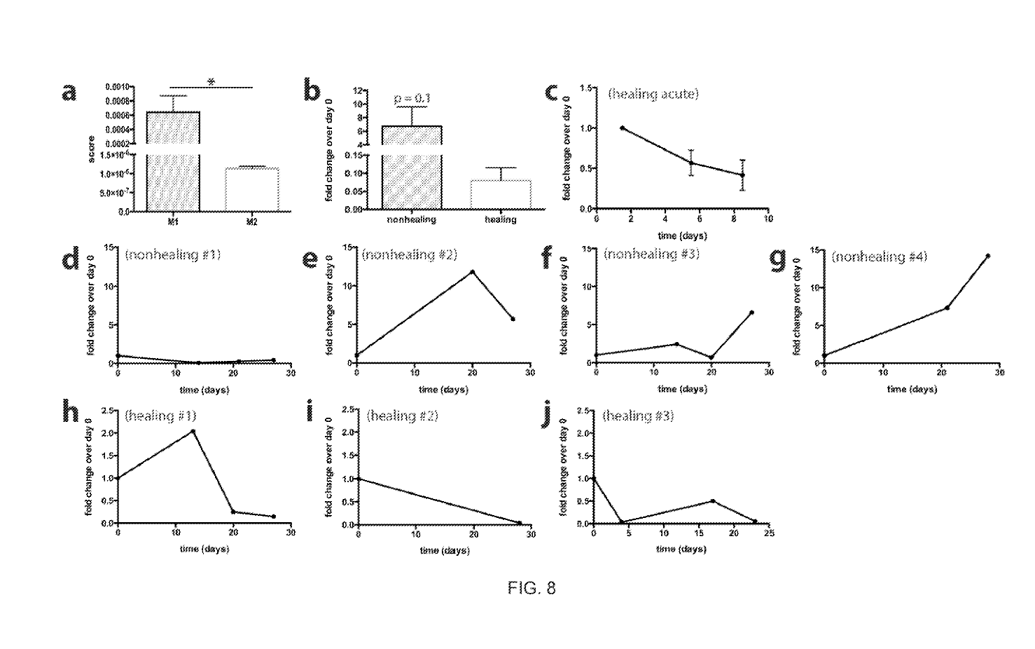

Results for the weighted scaling approach are depicted in FIG. 6.

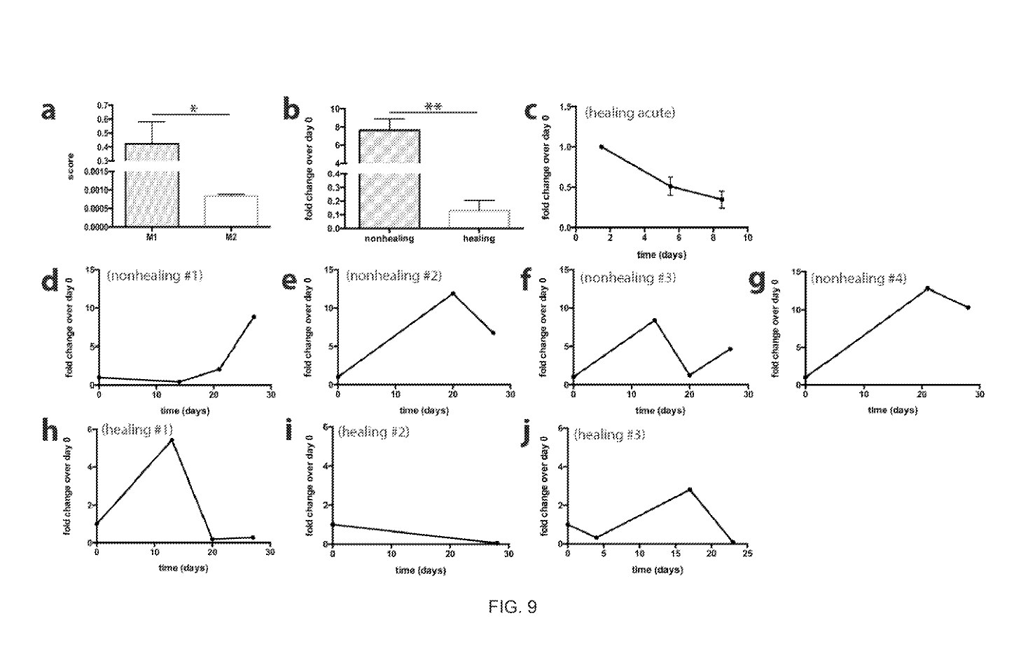

Results for the greedy approach are depicted in FIG. 7.

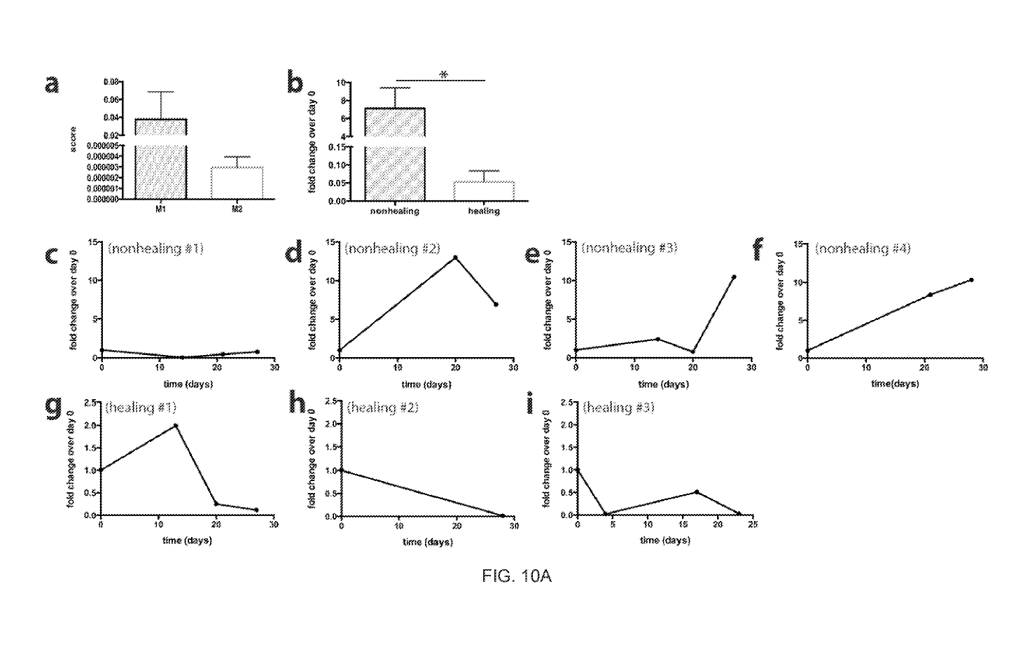

Results for the mean-centering approach are depicted in FIG. 8.

To address the question of whether those higher-expressed genes are more important contributors to the M1/M2 score, the opposite of mean-centering method was performed by simply summing the contributions from each gene thereby allowing contribution from all the genes analyzed based on their inherent levels of expression as depicted in FIG. 9.

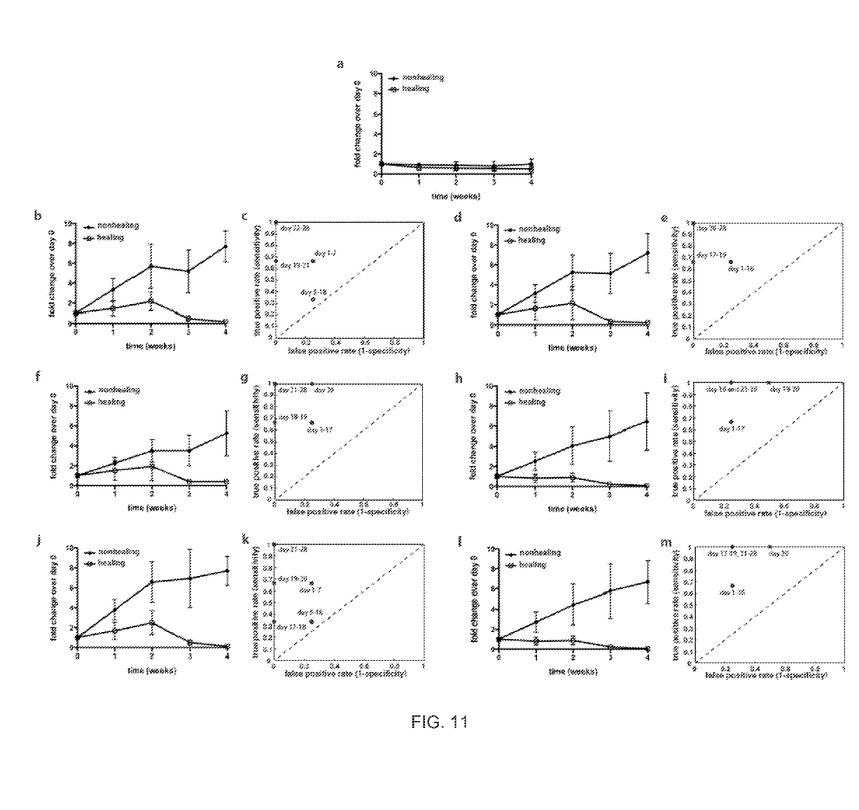

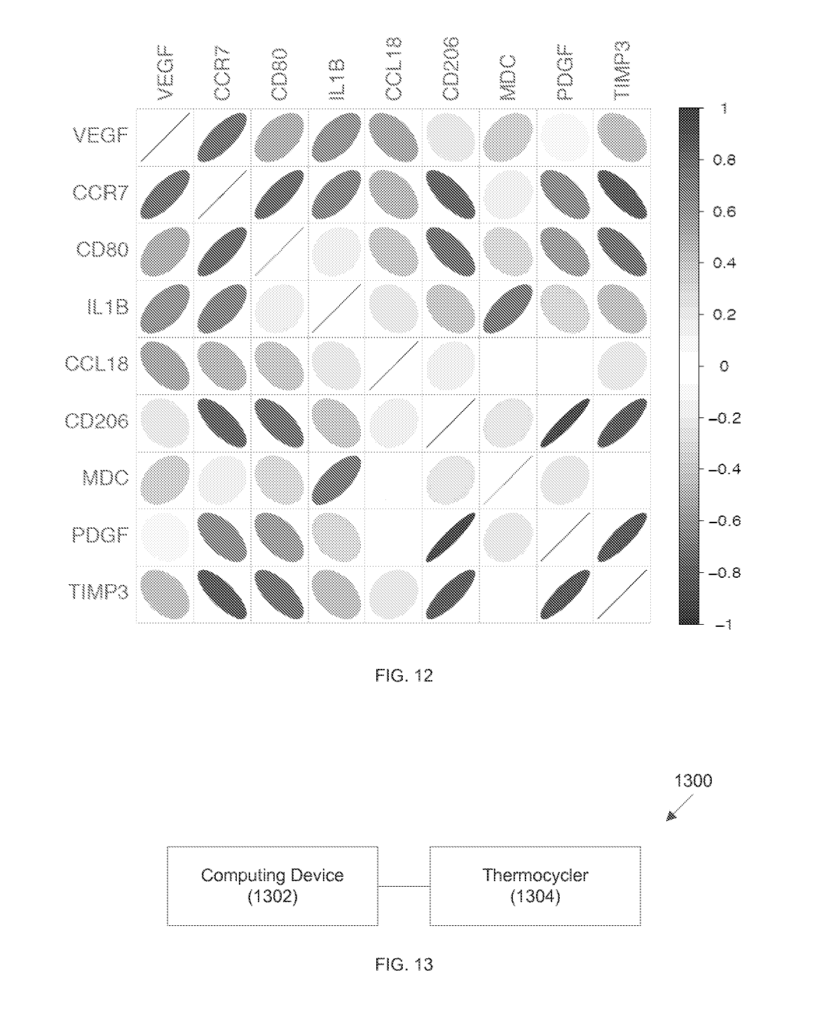

Lastly, with the aim of reducing the number of genes even further and to determine the major M1 and M2 contributors to the predictive M1/M2 score, one M1 and one M2 marker was chosen to define the M1/M2 score. In the case of a large sample size, a number of methodical approaches exist for feature selection. The small sample size prevented implementation of these methods. However, it was hypothesized that out of all possible combinations of M1/M2, those with a highly negatively correlated M1 and M2 genes would most likely yield the best outcome. To this end, a correlation matrix of the in vitro dataset was calculated and is depicted in FIG. 12. Screening for M1/M2 combinations with a cut off point of R<−0.3, IL1B/CD206 was found to accurately describe healing as depicted in FIG. 10A.

CD163 is another marker for a subtype of M2 macrophages referred to as M2c. IL1B/CD163 was also found to accurately describe healing as depicted in FIG. 10B.

In order to assess application of each M1/M2 score in predicting healing outcomes, and to compare the proposed methods to one another, profile analysis was performed on each method and the corresponding true positive rate was plotted versus false positive rate over the course of 4 weeks (FIG. 10). Profile analysis revealed that the difference between healing and nonhealing wounds becomes significant over time, with 3 out of 6 methods accurately predicting healing outcomes as early as 3 weeks after initial visit. Although promising, for robust measurement of the diagnostic application of these methods, the results need to be verified in studies with larger sample size using conventional assessments such as ROC curves to find an M1/M2 threshold that is of clinical relevance. Utility of each method as diagnostic at 4 weeks compared to wound size is summarized in Table 13.

TABLE 13
Utility of each method as diagnostic compared to wound size. True
positive rate, true negative rate, positive predictive value, negative
predictive value, and accuracy are reported at 4 weeks after initial
visit using 1 as the threshold for M1/M2 score.

| | | True | Positive | | |
|---|---|---|---|---|---|
| | True Positive | Negative | Predictive | Negative | |
| | Rate | Rate | Value | Predictive | Accu- |
| | (sensitivity) | (specificity) | (precision) | Value | racy |
| Wound | 66 | 50 | 50 | 66 | 57 |
| size | | | | | |
| PCA | 100 | 100 | 100 | 100 | 100 |
| Weighted | 100 | 100 | 100 | 100 | 100 |
| scaling | | | | | |
| Greedy | 100 | 100 | 100 | 100 | 100 |
| Mean- | 100 | 75 | 75 | 100 | 86 |
| centering | | | | | |
| Linear | 100 | 100 | 100 | 100 | 100 |
| sum | | | | | |
| IL1B/ | 100 | 75 | 75 | 100 | 86 |
| CD206 | | | | | |
| IL1B/ | 80 | 100 | 100 | 83 | 90 |
| CD163 | | | | | |

### Application of M1/M2 Ratios to in Vitro Testing of Biomaterials

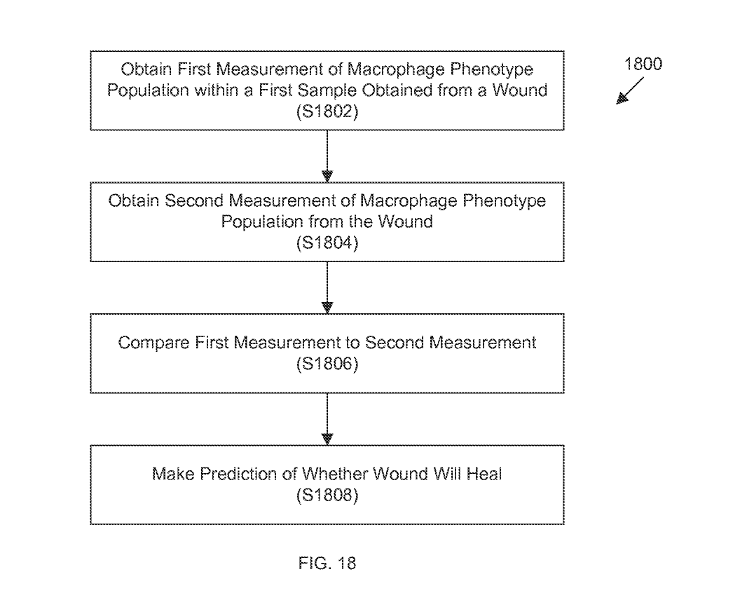

Referring now to FIG. 14, in vivo testing of 4 different biomaterials designed to be used as bone scaffolds for bone repair and regeneration indicated that interferon gamma (IFNg) material induced more vascularization than other materials. Considering the importance of macrophages for the healing of all tissues including bone, it was hypothesized that the material that yielded the most vascularization in vivo (i.e., IFNg material) would induce an effective M1-to-M2 transition in vitro.

To test this hypothesis, undifferentiated macrophages were cultured on the 4 different scaffolds in vitro and analyzed for expression of known M1 and M2 genes after 6 days of culture. Using the proposed “linear sum” method, an M1/M2 score was calculated for each scaffold. Comparison of the M1/M2 score between different materials revealed that in agreement with the hypothesis, IFNg material exhibited an initial increase followed by a decrease in the M1/M2 score between day 2 and day 6, suggesting an effective M1-to-M2 transition of macrophages over time. Without this score, the number of genes analyzed makes the data extremely difficult to interpret as seen in FIG. 6 of Kara L. Spiller et al., “Sequential delivery of immunomodulatory cytokines to facilitate the M1-to-M2 transition of macrophages and enhance vascularization of bone scaffolds,” 37 Biomaterials 194-207 (2015).

### Application of M1/M2 Ratios to Characterize Macrophage Behavior After Stent Implantation

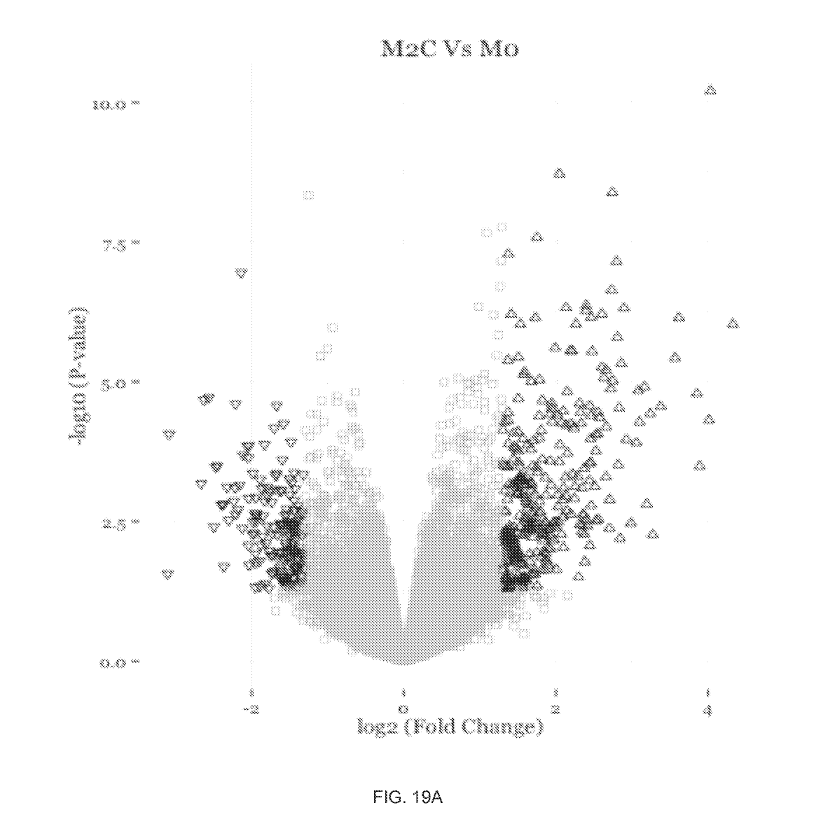

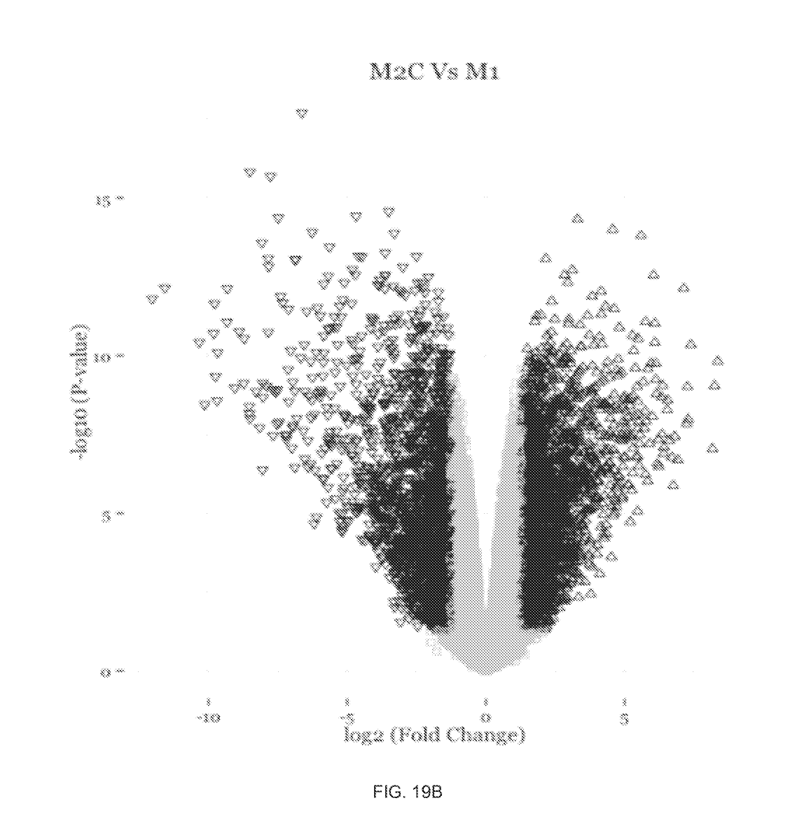

Implantation of the stents induces injury at the site of implantation. Considering the key role of macrophages in tissue repair and regeneration, and given the fact that M1 macrophages are dominant in the early inflammatory stages of wound healing and M2 macrophages are dominant in later stages of wound healing such as proliferation and remodeling, it was hypothesized that macrophages would exhibit a natural M1-to-M2 transition after stent implantation in rat arteries. Referring now to FIG. 15, expression profiling of macrophage markers using the proposed “linear sum” method revealed a decrease in the M1/M2 score over time, corroborating the hypothesis. Without this scoring method, the raw data (depicted in FIG. 16) is impossible to interpret.

### Predictive Power of Initial M1/M2 Ratio

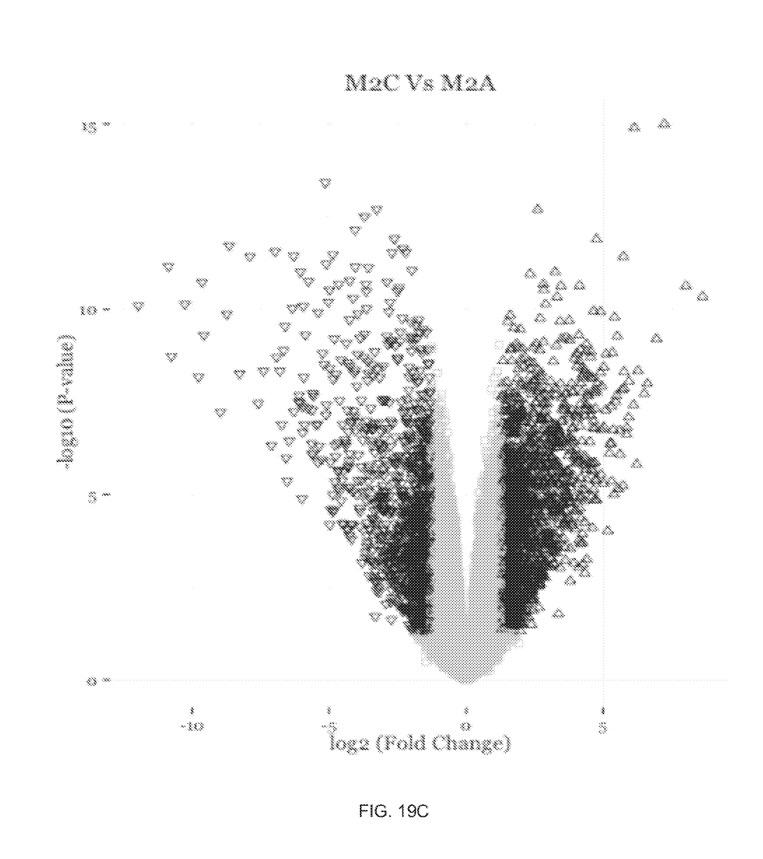

Referring to FIG. 17, the value of the M1/M2 score at the first sample collection was significantly higher for wounds that ultimately healed compared to those that did not (p<0.01, two-way ANOVA with Sidak post-hoc analysis, n=5). These results suggest that inflammation is beneficial for healing, which is supported by the clinical practice of wound debridement to stimulate inflammation and the contra-indication of anti-inflammatory treatments. Moreover, a delay in the administration of anti-inflammatory treatments after an initial pro-inflammatory period has been shown to be beneficial for healing in diabetic animal models. From a translational perspective, these results also suggest that this score might have the potential to identify those wounds that are more likely to respond to conservative treatment versus those that may benefit from a more aggressive approach.

### Evaluation of M1/M2 Ratios to Assess Effectiveness of Treatment of Nonhealing Wounds

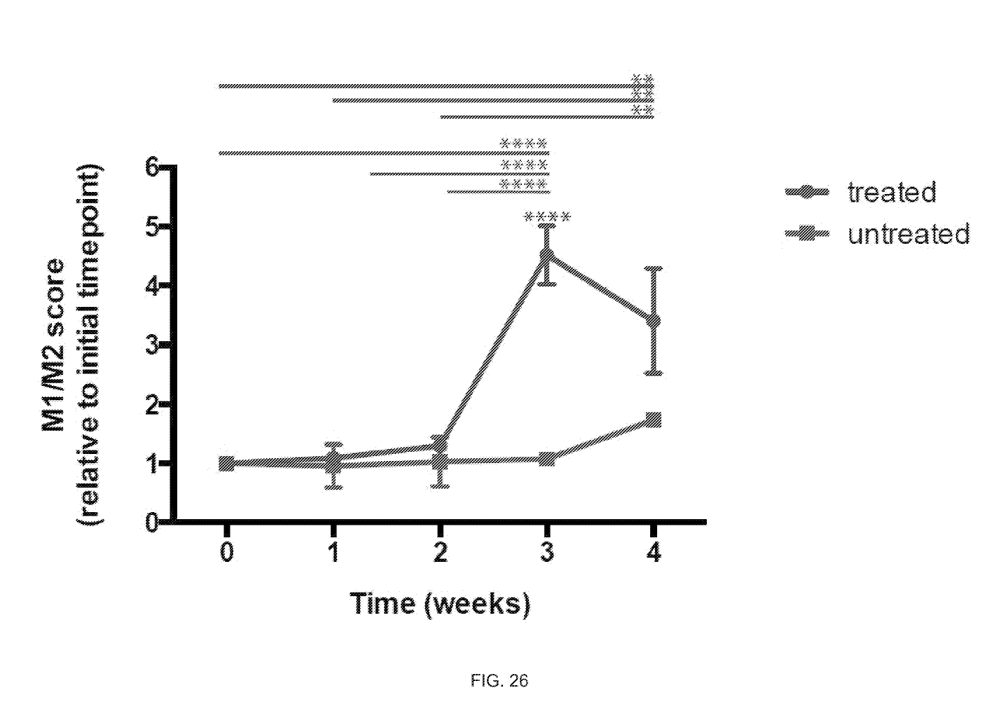

Referring now to FIG. 26 an M1/M2 score was calculated to compare the effect of ultrasound treatment on chronic diabetic ulcers. Low-intensity ultrasound treatment has been shown to be clinically effective in enhancing healing outcomes in chronic ulcers. However, the mechanism behind this technology is not yet fully understood. Applicant has previously shown that a macrophage-inspired gene expression ratio has potential to differentiate between healing and nonhealing ulcers. Moreover, change of this M1/M2 ratio over time in acute wounds is in agreement with the temporal dynamic of M1 and M2 macrophages found in normal wound healing, depicted by early expression of M1 markers transitioning into M2 markers at later time points. Applicant calculated the M1/M2 score to assess the effect of ultrasound treatment on chronic diabetic ulcers. As indicated in FIG. 26, the M1/M2 ratio did not change over time for the control group, accurately indicating non-healing. However, ultrasound treatment caused an increase in the M1/M2 score, which then decreased over time, ultimately resulting in healing. These results further support Applicant's findings that a transient increase in the M1/M2 score is beneficial for healing.

### Discussion

Taken together, the findings suggest that healing and nonhealing chronic diabetic ulcers are significantly different with respect to expression of M1 and M2 macrophage markers. Utilizing a number of methods to convert gene expression data into a combinatorial score that reflects the underlying physiology of wound healing, Applicant was able to use gene expression signature of in vitro polarized macrophages to indicate the inflammatory state of the wound. To the best of Applicant's knowledge, this study confirms for the first time that macrophages in human nonhealing diabetic wounds have a persistently elevated M1 character, while diabetic wounds that heal progress through a more natural M1-to-M2 transition. The results demonstrate that M1 and M2 macrophage gene expression signatures have the potential to be used as reference in quantification of wound healing progression, as well as prediction of healing outcomes.

Wound healing is a complex process and can be divided into several stages: hemostasis, inflammation, proliferation or granulation, and remodeling. Macrophages are key players in the onset and resolution of inflammation and are known to play critical roles in various stages of wound healing. Considering the fundamental role of macrophages in various stages of wound healing, and using the M1-to-M2 transitioning as an indication of tissue regeneration and healing, Applicant aimed to quantify the M1-to-M2 transition in chronic diabetic ulcer over time and to study its association with healing outcomes.

The conventional method for characterization of macrophage profile in biological tissue is immunohistochemistry (IHC). However, IHC approaches are extremely time-consuming, expensive, and only semi-quantitative. Although some of these limitations have been addressed in flow cytometry methods, practical challenges such as tissue digestion and small sampling volume still remain unresolved.

Applicant utilized gene expression of the wound tissue. Looking for gene expression enabled Applicant to consider using wound debrided tissue as the source of tissue. Using debrided wound tissue makes embodiments of the invention extremely advantageous over alternative methods that use optical approaches or wound fluid for assessment and quantification of wound healing progression. Such optical or fluid-based methods impose additional burdens both on the patient and on the care provider, whereas wound debridement is a procedure commonly performed as part of the standard wound care regimen. Moreover, optical or fluid-based methods suffer from high variability from patient to patient (not all wounds are exudative especially as they heal) as well as practical challenges such as detection methods. Moreover, such methods are also time consuming and expensive.

Embodiments of the invention described herein can also be used as a means of quantifying the effectiveness of an experimental therapy, which may be useful in facilitating regulatory approval of novel treatment strategies.

Applicant set out to convert gene expression data into a combinatorial score based on the underlying biology of M1-to-M2 transitioning of macrophages in the wound, using gene expression profile of in vitro polarized M1 and M2 macrophages. Because of the heterogeneity of debrided wound tissue, the total number of macrophages varies from sample to sample, which necessitates some form of data normalization before raw data can be used. Interestingly, defining a quotient of M1 markers over M2 markers, expression values are essentially normalized as the ratio of genes is independent of total number of cells.

Applicant then defined an M1/M2 score using six different methods to weigh M1 and M2 genes. In all methods, the M1/M2 score decreases over time in healing chronic diabetic ulcers, whereas it stays constant if not increases in nonhealing chronic diabetic ulcers. Applicant found this difference to be significant at 4 weeks, and already outperform the gold standard of the wound care, which is based on reduction in wound size. Moreover, Applicant found that decreasing trend of M1/M2 score in healing chronic diabetic ulcers resembles the trend observed in acute normal wounds, although with a much slower rate. Unlike wound size, using the M1/M2 score, healing and nonhealing chronic diabetic ulcers were found to be significantly different at 4 weeks, confirming that indeed healing and nonhealing wounds are different with respect to expression of M1 and M2 macrophage markers. Another interesting finding was the results obtained from linear sum method. Despite common belief that without normalization genes with higher expression values would dominate the score and mask the effect of other genes, Applicant found this method to effectively differentiate between healing and nonhealing diabetic wounds. This could be indicative of the importance of the genes with higher expression values in the wound healing process, something that need to be verified in future studies. Similarly, although Applicant found IL1B over CD206 to successfully differentiate between healing and nonhealing wounds at 4 weeks, this finding needs to be verified in independent studies due to possibility of over-fitting because, unlike other methods, definition of the model was based on the data.

By choosing 9 key genes that describe macrophage phenotype, normalization of M1 to M2 genes, and comparison of the score back to a baseline value, Applicant was able to track macrophage behavior using a method that is insensitive to patient-to-patient variability, wound heterogeneity, and variability in sampling methods. It has been suggested that a more accurate method of assessing wound progression would save an average of $12,600 per patient if ineffective treatments could be discontinued sooner. Remarkably, despite the small sample size of this study, Applicant found highly significant differences between changes in M1 over M2 scores for healing and nonhealing diabetic ulcers, suggesting a potential for its use as a diagnostic.

Although the sample size did not allow for thorough assessment of the predictive functionality of the proposed methods, Applicant compared the methods over time to one another and to wound size. Given that debrided tissue was used as the tissue source, and since wound debridement is already a standard part of wound care, this approach has great potential to be easily incorporated in wound care regimen. Although preliminary at this point, the results suggest that a small subset of genes can be used to define a macrophage signature, which in return facilitates incorporation of these method as an off-site qRT-PCR based diagnostic assay. Alternatively, with the advent of portable gene sequencing technologies, Applicant envisions this method for real-time measurement of the wound healing progression to complement physician's assessment and discretion in the clinic.

Taken together, Applicant's results suggest that macrophage gene expression signature may be strongly associated with wound healing progression and has the potential to be used in monitoring wound healing progression and to provide diagnostic information on healing outcomes. Furthermore, these findings shed light on the promise of using macrophage gene expression signatures to explore existing gene expression profiles of wounds, as well as other tissues. Given the importance of macrophages in the function and dysfunction of all tissues, the novel techniques described herein may be useful for the study of macrophage behavior in other disease and injury situations.

### Equivalents

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A method of predicting whether a wound will heal, the method comprising:
obtaining a first measurement of a first macrophage phenotype population within a first sample obtained from a wound;
obtaining a second measurement of a second macrophage phenotype population from the wound, wherein the second measurement of the second macrophage phenotype population is either:
a different macrophage phenotype obtained from the first sample; or
the same macrophage phenotype obtained from a second, later sample from the wound;
comparing the first measurement to the second measurement; and
predicting whether the wound will heal based on a result of the comparing step.

2.-20. (canceled)

21. A method of assessing a sample, the method comprising:
calculating a first ratio of M1 macrophages to M2 macrophages in a first sample based on gene expression values for at least one marker associated with M1 macrophage activity and at least one marker associated with M2 macrophage activity.

22.-27. (canceled)

28. The method of claim 21, wherein the calculating step includes:
calculating a first function of gene expression values of each of a first plurality of markers associated with M1 macrophages; and
calculating a second function of gene expression values of each of a second plurality of markers associated with M2 macrophages.

29.-37. (canceled)

38. The method of claim 21, further comprising:
calculating a second ratio of M1 macrophages to M2 macrophages in a second sample based on gene expression values for at least one marker associated with M1 macrophage activity and at least one marker associated with M2 macrophage activity, the second sample obtained from a same source as the first sample after passage of a period of time; and
comparing the second ratio to the first ratio.

39.-51. (canceled)

52. A non-transitory computer readable medium containing computer-readable program code including instructions for performing the method of claim 1.

53. A system comprising:
a gene expression device; and
a processor programmed to implement the method of claim 1.

54. (canceled)

55. A method of assessing a wound, the method comprising:
extracting RNA from debrided wound tissue;
measuring expression of one or more genes within the RNA; and
calculating a ratio of M1 macrophages to M2 macrophages based on the measured gene expression.

56. The method of claim 55, wherein the debrided wound tissue was removed from a dressing previously applied a wound.

57. The method of claim 55, wherein the debrided wound tissue is from one or more selected from the group consisting of: a diabetic ulcer, a pressure ulcer, a chronic venous ulcer, a burn, a wound caused by an autoimmune disease, a wound caused by Crohn's disease, a wound caused by atherosclerosis, a tumor, a medical implant insertion point, a surgical wound, a bone fracture, a tissue tear, and a tissue rupture.

58. The method of claim 55, wherein the measuring expression step includes using one or more tools or techniques selected from the group consisting of: cDNA synthesis, quantitative PCR (qPCR), microarrays, and RNA Sequencing (RNA-seq).

59. A high-throughput screening system comprising:
a measurement device; and
a data processor programmed to implement the method of claim 1.

60. A method of monitoring effectiveness of a treatment of a non-healing wound or a tumor, the method comprising:
administering to a patient a therapeutic agent designed to treat a non-healing wound or a tumor;
obtaining a first measurement of a first macrophage phenotype population within a first sample obtained from the non-healing wound or the tumor;
obtaining a second measurement of second macrophage phenotype population from the non-healing wound or the tumor, wherein the second measurement of the second macrophage phenotype population is either:
a different macrophage phenotype obtained from the first sample or the tumor; or
the same macrophage phenotype obtained from a second, later sample from the non-healing wound or the tumor;
comparing the first measurement to the second measurement; and
assessing whether the treatment of the non-healing wound or the tumor is effective based on a result of comparing the measurements.

61. The method of claim 60, wherein the therapeutic agent is selected from the group consisting of an L-arginine, hyperbaric oxygen, a moist saline dressing, an isotonic sodium chloride gel, a hydroactive paste, a polyvinyl film dressing, a hydrocolloid dressing, a calcium alginate dressing, and a hydrofiber dressing.

62. The method of claim 60, wherein the treatment is low-intensity ultrasound treatment.

63. The method of claim 60, further comprising
comparing an M1/M2 ratio with a threshold value that discriminates between (i) wound healing and non healing or (ii) tumor progression and non-progression; and
adjusting the treatment based on the M1/M2 ratio, wherein:
if the M1/M2 ratio is at or below the threshold value, the administration of therapeutic agent is increased, and
if the M1/M2 ratio is above the threshold value, the administration of the therapeutic agent is not increased.

64. The method of claim 63, wherein if the level is at or below the threshold value, the therapeutic agent is replaced by a different therapeutic agent.

65. A method of treating a wound comprising:
administering an effective amount of interferon gamma (IFNg) to the wound.

66. A non-transitory computer readable medium containing computer-readable program code including instructions for performing the method of claim 21.

67. A system comprising:
a gene expression device; and
a processor programmed to implement the method of claim 21.

68. A high-throughput screening system comprising:
a measurement device; and
a data processor programmed to implement the method of claim 21.


SEQUENCE LISTING (24 sequences)

SEQ ID NO: 1 - DNA, 21 bp, Artificial Sequence
  Artificially synthesized
tgaggtcacggacgattacat

SEQ ID NO: 2 - DNA, 22 bp, Artificial Sequence
  Artificially synthesized
gtaggcccacgaaacaaatgat

SEQ ID NO: 3 - DNA, 21 bp, Artificial Sequence
  Artificially synthesized
aaactcgcatctactggcaaa

SEQ ID NO: 4 - DNA, 21 bp, Artificial Sequence
  Artificially synthesized
ggttcttgtactcgggccata

SEQ ID NO: 5 - DNA, 23 bp, Artificial Sequence
  Artificially synthesized
atgatggcttattacagtggcaa

SEQ ID NO: 6 - DNA, 20 bp, Artificial Sequence
  Artificially synthesized
gtcggagattcgtagctgga

SEQ ID NO: 7 - DNA, 20 bp, Artificial Sequence
  Artificially synthesized
gctctctgcccgtctatacc

SEQ ID NO: 8 - DNA, 24 bp, Artificial Sequence
  Artificially synthesized
gggctggtttcagaatagtcaact

SEQ ID NO: 9 - DNA, 23 bp, Artificial Sequence
  Artificially synthesized
tttgtcaacttgagtcccttcac

SEQ ID NO: 10 - DNA, 21 bp, Artificial Sequence
  Artificially synthesized
tcccgctacacttgttttcac

SEQ ID NO: 11 - DNA, 19 bp, Artificial Sequence
  Artificially synthesized
aaggcggtgacctcacaag

SEQ ID NO: 12 - DNA, 22 bp, Artificial Sequence
  Artificially synthesized
aaagtccaattcctcgatggtg

SEQ ID NO: 13 - DNA, 21 bp, Artificial Sequence
  Artificially synthesized
gcgtggtgttgctaaccttca

SEQ ID NO: 14 - DNA, 20 bp, Artificial Sequence
  Artificially synthesized
aaggccacggtcatcagagt

SEQ ID NO: 15 - DNA, 21 bp, Artificial Sequence
  Artificially synthesized
ctcgatccgctcctttgatga

SEQ ID NO: 16 - DNA, 19 bp, Artificial Sequence
  Artificially synthesized
cgttggtgcggtctatgag

SEQ ID NO: 17 - DNA, 20 bp, Artificial Sequence
  Artificially synthesized
accgaggcttcaccaagatg

SEQ ID NO: 18 - DNA, 21 bp, Artificial Sequence
  Artificially synthesized
catcatagacgcgacctgtca

SEQ ID NO: 19 - DNA, 21 bp, Artificial Sequence
  Artificially synthesized
gcgtggtgttgctaaccttca

SEQ ID NO: 20 - DNA, 20 bp, Artificial Sequence
  Artificially synthesized
aaggccacggtcatcagagt

SEQ ID NO: 21 - DNA, 19 bp, Artificial Sequence
  Artificially synthesized
aaggcggtgacctcacaag

SEQ ID NO: 22 - DNA, 22 bp, Artificial Sequence
  Artificially synthesized
aaagtccaattcctcgatggtg

SEQ ID NO: 23 - DNA, 23 bp, Artificial Sequence
  Artificially synthesized
tttgtcaacttgagtcccttcac

SEQ ID NO: 24 - DNA, 21 bp, Artificial Sequence
  Artificially synthesized
tcccgctacacttgttttcac